(19) United States
(12) Patent Application Publication
(10) Pub. No.: US 2024/0280558 A1
(43) Pub. Date: Aug. 22, 2024

(54) SYSTEMS AND METHODS FOR PROCESSING MASS SPECTROMETRY DATASETS

(71) Applicant: Seer, Inc., Redwood City, CA (US)

(72) Inventors: Theodore Platt, Danville, CA (US); Iman Mohtashemi, Mountain House, CA (US); Hugo Kitano, San Francisco, CA (US); Asim Siddiqui, San Francisco, CA (US)

(21) Appl. No.: 18/578,513

(22) PCT Filed: Jul. 13, 2022
(86) PCT No.: PCT/US2022/037003

§ 371 Date: Jan. 11, 2024

Related U.S. Application Data
(60) Provisional application No. 63/221,141, filed on Jul. 13, 2021.
(60) Provisional application No. 63/256,257, filed on Oct. 15, 2021.
(60) Provisional application No. 63/306,969, filed on Feb. 4, 2022.
(60) Provisional application No. 63/348,860, filed on Jun. 3, 2022.

(51) Int. Cl. G01N 33/487; G01N 30/86; G01N 33/68

(52) U.S. Cl. CPC G01N 33/48792; G01N 30/8631; G01N 33/6848

(57) ABSTRACT

In some aspects, the present disclosure provides a method for normalizing and processing mass spectrometry datasets. In some embodiments, the method comprises loading a plurality of mass spectrometry data obtained from a plurality of samples into a memory of a computing node to generate a cached dataset. In some embodiments, the method comprises transmitting a copy of the cached dataset to a plurality of cache memories of a plurality of computing nodes. In some embodiments, the method comprises determining, using the plurality of computing nodes, a plurality of feature values for the plurality of mass spectrometry data. In some embodiments, the method comprises normalizing, using the plurality of computing nodes, across the plurality of mass spectrometry datasets using the plurality of feature values to generate a plurality of normalized mass spectrometry data.

### CROSS-REFERENCE

This application claims the benefit of U.S. Provisional Application No. 63/221,141, filed Jul. 13, 2021, U.S. Provisional Application No. 63/256,257, filed Oct. 15, 2021, U.S. Provisional Application No. 63/306,969, filed Feb. 4, 2022, and U.S. Provisional Application No. 63/348,860, filed Jun. 3, 2022, each of which are incorporated herein by reference in their entirety.

### BACKGROUND

Biological samples contain a wide variety of proteins and nucleic acids. Computational methods are needed for elucidating the presence and concentration of proteins and nucleic acids as well as any correlations between proteins and nucleic acids that may be indicative of a biological state.

### SUMMARY

In some aspects, the present disclosure provides a computer-implemented method for normalizing and processing mass spectrometry datasets, comprising: (a) obtaining a plurality of mass spectrometry datasets obtained from a plurality of samples; (b) loading the plurality of mass spectrometry datasets into a memory of a computing node to generate a cached dataset; (c) transmitting a copy of the cached dataset to a plurality of cache memories of a plurality of computing nodes; (d) determining, using the plurality of computing nodes, a plurality of feature values for the plurality of mass spectrometry datasets; (e) normalizing, using the plurality of computing nodes, across the plurality of mass spectrometry datasets using the plurality of feature values to generate a plurality of normalized mass spectrometry datasets; and (f) processing the plurality of normalized mass spectrometry datasets to compare the plurality of samples.

In some embodiments, the plurality of mass spectrometry datasets comprises a set of precursors for each sample in the plurality of samples.

In some embodiments, the set of precursors comprises a set of biomolecule precursors.

In some embodiments, the set of biomolecule precursors comprises a set of polyamino acid precursors.

In some embodiments, the plurality of mass spectrometry datasets comprises a set of chemical identifications for each sample in the plurality of samples.

In some embodiments, the set of chemical identifications comprises a set of biomolecule identifications.

In some embodiments, the set of biomolecule identifications comprises a set of polyamino acid identifications.

In some embodiments, the set of polyamino acid identifications comprises a set of tryptic or semi-tryptic peptide identifications.

In some embodiments, the plurality of mass spectrometry datasets comprises a set of chemical intensities for each sample in the plurality of samples.

In some embodiments, the set of chemical intensities comprises a set of biomolecule intensities.

In some embodiments, the set of biomolecule intensities comprises a set of polyamino acid intensities.

In some embodiments, the set of polyamino acid intensities comprises a set of tryptic or semi-tryptic peptide intensities.

In some embodiments, the set of polyamino acid identifications comprises a set of protein group identifications.

In some embodiments, the set of polyamino acid intensities comprises a set of protein group intensities.

In some embodiments, the plurality of mass spectrometry datasets comprises a data independent acquisition (DIA) mass spectrometry dataset, a data dependent acquisition (DDA) mass spectrometry dataset, or both.

In some embodiments, the plurality of mass spectrometry datasets comprises a LC-MS dataset, a LC-MS/MS dataset, or both.

In some embodiments, the plurality of samples comprises at least 500, 5000, or 50000 samples.

In some embodiments, the plurality of samples comprises at most 5000, 50000, 500000 samples.

In some embodiments, the plurality of samples comprises a complex sample.

In some embodiments, the complex sample comprises a biological sample.

In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises plasma or serum.

In some embodiments, the complex sample comprises at least 100, 1000, 10000, 100000, or 1000000 unique biomolecules.

In some embodiments, the complex sample comprises at least 100, 1000, 10000, 100000, or 1000000 unique proteins.

In some embodiments, the complex sample comprises at most 1000, 10000, 100000, 1000000, or 10000000 unique biomolecules.

In some embodiments, the complex sample comprises at most 1000, 10000, 100000, 1000000, or 10000000 unique proteins.

In some embodiments, the complex sample comprises a biomolecule comprising at least about 0.1, 1, 10, 100, or 1000 kiloDaltons (kDa) in molecular weight.

In some embodiments, the complex sample comprises a biomolecule comprising at most about 1, 10, 100, 1000, or 10000 kiloDaltons (kDa) in molecular weight.

In some embodiments, the feature values are based on isotopic clusters.

In some embodiments, the feature values comprise retention time, mass-to-charge ratio, aggregate peak area of the isotope cluster, ion mobility, or any combination thereof.

In some embodiments, the normalizing generates a set of aligned precursors for each mass spectrometry dataset in the plurality of mass spectrometry datasets.

In some embodiments, the computer-implemented method further comprises identifying a first chemical from a first mass spectrometry dataset in the plurality of mass spectrometry datasets based on an aligned precursor in the set of aligned precursors of a second mass spectrometry dataset.

In some embodiments, the plurality of feature values comprises a feature value for the set of precursors of each mass spectrometry dataset in the plurality of mass spectrometry datasets.

In some embodiments, the feature value is configured for normalizing retention time, mass-to-charge ratio, ion mobility, or a combination thereof.

In some embodiments, the feature value is a shifting value.

In some embodiments, the determining comprises minimizing an objective function, using a computing node in the plurality of computing nodes, based on a pair of mass spectrometry datasets in the plurality of mass spectrometry datasets.

In some embodiments, the determining comprises minimizing the objective function for a unique pair of mass spectrometry datasets in the plurality of mass spectrometry datasets for each computing node in the plurality of computing nodes.

In some embodiments, the normalizing generates a set of relative abundances for each mass spectrometry dataset in the plurality of mass spectrometry datasets.

In some embodiments, normalizing comprises label-free quantification.

In some embodiments, the set of relative abundances comprises a set of chemical relative abundances.

In some embodiments, the set of chemical relative abundances comprises a set of biomolecule relative abundances.

In some embodiments, the set of biomolecule relative abundances comprises a set of polyamino acid relative abundances.

In some embodiments, the set of relative abundances represent relative abundances of chemicals between the plurality of mass spectrometry datasets.

In some embodiments, the set of relative abundances represent relative abundances of polyamino acids between the plurality of mass spectrometry datasets.

In some embodiments, the plurality of feature values comprises a feature value for the set of chemical intensities of each mass spectrometry dataset in the plurality of mass spectrometry datasets.

In some embodiments, the normalizing comprises adjusting the set of chemical intensities for each mass spectrometry dataset in the plurality of mass spectrometry datasets based on the plurality of feature values.

In some embodiments, the determining comprises minimizing an objective function, using a computing node in the plurality of computing nodes, based on a pair of mass spectrometry datasets in the plurality of mass spectrometry datasets.

In some embodiments, the determining comprises minimizing the objective function for a unique pair of mass spectrometry datasets in the plurality of mass spectrometry datasets for each computing node in the plurality of computing nodes.

In some embodiments, the objective function comprises:

$$L = {\sum }_{p}^{N} \vert \frac{I ( {Norm}_{A} , p )}{I ( {Norm}_{B} , p )} \vert ,$$

wherein N is a number of chemical identifications in the set of chemical identifications, wherein p is a chemical in the set of chemical identifications, wherein I is an intensity value for the set of chemical intensities, wherein Norm_{A} is a first feature value for a first mass spectrometry dataset in the pair of mass spectrometry datasets, and wherein Norm_{B} is a second feature value for a second mass spectrometry dataset in the pair of mass spectrometry datasets.

In some embodiments, the objective function comprises:

$$L = {\sum }_{A , B}^{M} {\sum }_{p}^{N} \vert \frac{I ( {Norm}_{A} , p , A )}{I ( {Norm}_{B} , p , B )} \vert ,$$

wherein M is a number of unique pairs of mass spectrometry datasets in the plurality of mass spectrometry datasets, and wherein A,B is the unique pair of mass spectrometry datasets in the plurality of mass spectrometry datasets.

In some embodiments, the normalizing generates a set of chemical identifications for each mass spectrometry dataset in the plurality of mass spectrometry datasets.

In some embodiments, the set of chemical identifications comprises a set of protein group identifications.

In some embodiments, the normalizing comprises assigning a first peptide identification in a first mass spectrometry dataset in the plurality of mass spectrometry datasets and a second peptide identification in a second mass spectrometry dataset in the plurality of mass spectrometry datasets to the same protein group.

In some embodiments, the determining comprises minimizing an objective function, using a computing node in the plurality of computing nodes, based on a pair of mass spectrometry datasets in the plurality of mass spectrometry datasets.

In some embodiments, the determining comprises minimizing the objective function a unique pair of mass spectrometry datasets in the plurality of mass spectrometry datasets for each computing node in the plurality of computing nodes.

In some embodiments, a processing time for performing (b)-(f) is substantially linear as a function of a number of mass spectrometry datasets in the plurality of mass spectrometry datasets.

In some embodiments, performing (b)-(f) takes less than ax^{1.8} amount of compute time, wherein x is a number of mass spectrometry datasets in the plurality of mass spectrometry datasets, and wherein a is a constant.

In some embodiments, performing (b)-(f) takes less than ax^{1.6} amount of compute time, wherein x is a number of mass spectrometry datasets in the plurality of mass spectrometry datasets, and wherein a is a constant.

In some embodiments, performing (b)-(f) takes less than ax^{1.4} amount of compute time, wherein x is a number of mass spectrometry datasets in the plurality of mass spectrometry datasets, and wherein a is a constant.

In some embodiments, performing (b)-(f) takes less than ax^{1.2} amount of compute time, wherein x is a number of mass spectrometry datasets in the plurality of mass spectrometry datasets, and wherein a is a constant.

In some embodiments, performing (b)-(f) takes less than ax amount of compute time, wherein x is a number of mass spectrometry datasets in the plurality of mass spectrometry datasets, and wherein a is a constant.

In some embodiments, the processing further comprises determining a biomarker based on the plurality of normalized mass spectrometry datasets.

In some embodiments, the processing further comprises performing a power curve analysis based on the plurality of normalized mass spectrometry datasets.

In some embodiments, the processing further comprises training a machine learning model based on the plurality of normalized mass spectrometry datasets.

In some embodiments, the processing further comprises performing clustering analysis based on the plurality of normalized mass spectrometry datasets.

In some embodiments, the computer-implemented method further comprises, before (a), performing a plurality of assays on the plurality of samples to generate the plurality of mass spectrometry datasets.

In some embodiments, the plurality of assays comprises selectively enriching a plurality of chemicals in the plurality of samples.

In some embodiments, the selectively enriching comprises contacting the plurality of samples with a surface.

In some embodiments, the surface comprises a particle surface of a particle.

In some embodiments, the particle comprises a paramagnetic core.

In some embodiments, the selectively enriching comprises contacting the plurality of samples with a plurality of surfaces comprising distinct surface chemistries.

In some embodiments, the contacting adsorbs the plurality of chemicals on the surface.

In some embodiments, the plurality of chemicals comprises a dynamic range of at least about 1, 2, 3, 4, 5, 6, 7, 8, 9, 10, 11, 12, 13, 14, 15, 16, 17, 18, or 19.

In some embodiments, the plurality of chemicals comprises a dynamic range of at most about 1, 2, 3, 4, 5, 6, 7, 8, 9, 10, 11, 12, 13, 14, 15, 16, 17, 18, or 19.

In some embodiments, the plurality of chemicals, when adsorbed, comprises a dynamic range that is decreased by at least about 1, 2, 3, 4, 5, 6, 7, 8, 9, 10, 11, 12, 13, 14, or 15 magnitudes.

In some embodiments, the selectively enriching comprises releasing the plurality of chemicals from the surface.

In some embodiments, the plurality of assays comprises performing mass spectrometry on the plurality of samples.

In some embodiments, the computing node is a local computing node.

In some embodiments, the plurality of computing nodes comprises at least 2, 5, 10, 100, 1000, 10000, or 100000 computing nodes.

In some embodiments, the plurality of computing nodes comprises at most 10, 100, 1000, 10000, 100000, or 1000000 computing nodes.

In some embodiments, the computing node is a cloud-computing node.

In some embodiments, the plurality of computing nodes is a plurality of cloud-computing nodes.

In some embodiments, the memory is a cache memory.

In some embodiments, the cached dataset is an unserialized cached dataset.

In some embodiments, the unserialized cached dataset is serialized to generate a serialized cached dataset.

In some embodiments, the serialized cached dataset is subdivided to generate a subdivided cached dataset.

In some embodiments, the copy of the cached dataset is a copy of at least a portion of the subdivided cached dataset.

In some embodiments, the transmitting comprises assembling a copy of at least a portion of the serialized cached dataset from the copy of the at least the portion of the subdivided cached dataset.

In some embodiments, the cached dataset comprises a pair of mass spectrometry datasets in the plurality of mass spectrometry datasets.

In some embodiments, the transmitting comprises transmitting, to each computing node in the plurality of nodes, a plurality of cached datasets each comprising a unique pair of mass spectrometry datasets in the plurality of mass spectrometry datasets.

In some embodiments, the copy of the cached dataset is shared by the plurality of computing nodes.

In some embodiments, the plurality of mass spectrometry datasets comprises a plurality of formats.

In some embodiments, the computer-implemented method further comprises, before (b), generating a harmonized plurality of mass spectrometry datasets comprising a harmonized format based on the plurality of mass spectrometry datasets.

In some embodiments, the loading comprises loading the harmonized plurality of mass spectrometry datasets to generate the cached dataset.

In some embodiments, the computer-implemented method further comprises, before (b), subdividing each harmonized mass spectrometry datasets in the plurality of mass spectrometry datasets to generate a plurality of mass spectrometry scans.

In some embodiments, the loading comprises loading the plurality of mass spectrometry scans to generate the cached dataset.

In some embodiments, the harmonized format comprises a compressed format.

In some embodiments, the harmonized format comprises a hierarchical format.

In some embodiments, the harmonized format comprises (i) the plurality of mass spectrometry datasets in an indexed series and (ii) indices of the indexed series.

In some embodiments, a mass spectrometry dataset in the plurality of mass spectrometry datasets comprises a different number of mass spectrometry scans compared to another mass spectrometry dataset in the plurality of mass spectrometry datasets.

In some embodiments, the harmonized format is capable of being read in arbitrary slides in the indexed series.

In some embodiments, the harmonized format is capable of inserting new datasets and/or being modified between arbitrary indices in the indexed series.

In some aspects, the present disclosure provides a computer-implemented method for performing a plurality of polyamino acid searches based on a plurality of mass spectra and a plurality of user specifications, comprising: (a) displaying a graphical user interface (GUI) to one or more users, wherein the GUI comprises (i) a first menu comprising a plurality of mass spectrum acquisition modes and (ii) a second menu comprising a plurality of mass spectrum search modes; (b) receiving the plurality of user specifications from the one or more users via the GUI, wherein each user specification in the plurality of user specifications comprises (i) a mass spectrum acquisition mode in the plurality of mass spectrum acquisition modes from the first menu and (ii) a mass spectrum search mode in the plurality of mass spectrum search modes from the second menu; (c) receiving the plurality of mass spectra from the one or more users, wherein the plurality of mass spectra comprises a plurality of formats; (d) generating a harmonized plurality of mass spectra based on the plurality of mass spectra and the plurality of formats, wherein the harmonized plurality of mass spectra comprises a harmonized format; and (e) performing the plurality of polyamino acid searches for each mass spectrum in the harmonized plurality of mass spectra based on the plurality of user specifications to generate a plurality of polyamino acid identifications.

In some embodiments, the plurality of mass spectrum acquisition modes comprises data independent acquisition (DIA) and data dependent acquisition (DDA).

In some embodiments, the plurality of mass spectrum search modes comprises a plurality of DIA search modes.

In some embodiments, the plurality of mass spectrum search modes comprises a plurality of DDA search modes.

In some embodiments, the computer-implemented method further comprises performing protein grouping based on the plurality of polyamino acid identifications to generate a plurality of protein groups.

In some embodiments, the computer-implemented method further comprises displaying a plurality of performance metrics for the plurality of polyamino acid searches, wherein the plurality of performance metrics comprises: (i) a plurality of peptide counts for each mass spectrum in the plurality of mass spectra and (ii) a plurality of protein group counts each mass spectrum in for the plurality of mass spectra.

In some embodiments, the plurality of performance metrics comprises a miscleavage rate for each mass spectrum in the plurality of mass spectra.

In some embodiments, the performing comprises: (a) subdividing each mass spectrum in the plurality of mass spectra to generate a plurality of mass spectrometry scans; (b) distributing the plurality of mass spectrometry scans onto a plurality of computing nodes; and (c) performing the plurality of polyamino acid searches, using the plurality of computing nodes, to generate the plurality of polyamino acid identifications.

In some embodiments, each mass spectrometry scan in the plurality of mass spectrometry scans comprises a plurality of intensities for a plurality of retention times.

In some embodiments, a first mass spectrometry scan in the plurality of mass spectrometry scans comprises a different mass-to-charge ratio compared to a second mass spectrometry scan in the plurality of mass spectrometry scans.

In some embodiments, the computer-implemented method further comprises performing mass spectrometry on a plurality of biological samples to generate the plurality of mass spectra.

In some embodiments, the generating further comprises transmitting a first polyamino acid identification of the plurality of polyamino acid identifications from a first computing node in the plurality of computing nodes to a second computing node in the plurality of computing nodes to identify a second polyamino acid identification of the plurality of polyamino acid identifications in the second computing node, wherein the first polyamino acid identification and the second polyamino acid identification are the same.

In some embodiments, the generating further comprises transmitting a probability value associated with a protein group assignment for a polyamino acid identification in the plurality of polyamino acid identifications from a first computing node in the plurality of computing nodes to a second computing node in the plurality of computing nodes.

In some embodiments, the plurality of computing nodes is a plurality of cloud-computing nodes.

In some embodiments, the plurality of cloud-computing nodes forms one or more computing clusters.

In some embodiments, the one or more computing clusters are high-performance computing (HPC) clusters.

In some embodiments, the plurality of cloud-computing nodes forms one or more virtual computing nodes.

In some aspects, the present disclosure provides a computer-implemented method for performing a plurality of polyamino acid searches based on a plurality of mass spectra and a plurality of user specifications, comprising: (a) receiving the plurality of user specifications from the one or more users via a GUI; (b) receiving the plurality of mass spectra from the one or more users, wherein the plurality of mass spectra comprises a plurality of formats; (c) generating a harmonized plurality of mass spectra based on the plurality of mass spectra and the plurality of formats, wherein the harmonized plurality of mass spectra comprises a harmonized format; and (d) performing the plurality of polyamino acid searches for each mass spectrum in the harmonized plurality of mass spectra based on the plurality of user specifications to generate a plurality of polyamino acid identifications.

In some aspects, the present disclosure provides a computer-implemented system for storing mass spectrometry datasets on a cloud platform, comprising: at least one digital processing device comprising: at least one processor, an operating system configured to perform executable instructions, a memory, and a computer program including instructions that, upon execution by the at least one processor, cause the at least one processor to perform at least: generating an event signal when a mass spectrometry dataset is received by the computer-implemented system, wherein the mass spectrometry dataset comprises at least one of a plurality of formats; triggering an event signal, wherein the event signal instantiates a serverless cloud computing instance; performing a data processing routine using the serverless cloud computing instance, wherein the data processing routine comprises: generating a harmonized mass spectrometry dataset comprising a harmonized data format based on the mass spectrometry dataset; and storing the harmonized mass spectrometry dataset on a storage system.

In some embodiments, the storage system comprises an object-based storage system, a distributed storage system, or an object-based distributed storage system.

In some embodiments, the harmonized mass spectrometry dataset comprises a columnar format.

In some embodiments, the instructions further comprise performing the data processing routine using a server cloud computing instance when the serverless cloud computing instance cannot be instantiated.

In some embodiments, the data processing routine further comprises (i) performing a plurality of polyamino acid searches based on the harmonized mass spectrometry dataset and a data acquisition mode of the mass spectrometry dataset to generate a plurality of polyamino acid identifications, and (ii) storing the plurality of polyamino acid identifications on the object-based storage system.

In some embodiments, the mass spectrometry dataset comprises at least one of a plurality of acquisition modes.

In some embodiments, the plurality of acquisition modes comprises data independent acquisition (DIA) and data dependent acquisition (DDA).

In some embodiments, the plurality of polyamino acid searches use a plurality of search modes.

In some embodiments, the plurality of search modes comprises a plurality of DIA search modes.

In some embodiments, the plurality of search modes comprises a plurality of DDA search modes.

In some embodiments, the data processing routine further comprises performing protein grouping based on the plurality of polyamino acid identifications to generate a plurality of protein groups.

In some embodiments, the performing the protein grouping comprises: (i) subdividing the harmonized mass spectrometry dataset to generate a plurality of mass spectrometry scans; (ii) distributing the plurality of mass spectrometry scans onto a plurality of computing nodes; and (iii) performing the plurality of polyamino acid searches, using the plurality of computing nodes, to generate the plurality of protein groups.

In some embodiments, each mass spectrometry scan in the plurality of mass spectrometry scans comprises a plurality of intensities for a plurality of retention times.

In some aspects, the present disclosure provides a computer-implemented method for storing mass spectrometry datasets on a cloud platform, comprising: (a) receiving a mass spectrometry dataset, wherein the mass spectrometry dataset comprises at least one of a plurality of formats; (b) generating an event signal based on the mass spectrometry dataset; (c) instantiating a serverless cloud computing instance based on the event signal; (d) performing a data processing routine using the serverless cloud computing instance, wherein the data processing routine comprises: (i) generating a harmonized mass spectrometry dataset comprising a harmonized data format based on the mass spectrometry dataset; and (ii) storing the harmonized mass spectrometry dataset on an object-based storage system.

In some embodiments, the harmonized mass spectrometry dataset comprises a columnar format.

In some embodiments, the computer-implemented method further comprises performing the data processing routine using a server cloud computing instance when the serverless cloud computing instance cannot be instantiated.

In some embodiments, the data processing routine further comprises (i) performing a plurality of polyamino acid searches based on the harmonized mass spectrometry dataset and a data acquisition mode of the mass spectrometry dataset to generate a plurality of polyamino acid identifications, and (ii) storing the plurality of polyamino acid identifications on the object-based storage system.

In some embodiments, the mass spectrometry dataset comprises at least one of a plurality of acquisition modes.

In some embodiments, the plurality of acquisition modes comprises data independent acquisition (DIA) and data dependent acquisition (DDA).

In some embodiments, the plurality of polyamino acid searches use a plurality of search modes.

In some embodiments, the plurality of search modes comprises a plurality of DIA search modes.

In some embodiments, the plurality of search modes comprises a plurality of DDA search modes.

In some embodiments, the data processing routine further comprises performing protein grouping based on the plurality of polyamino acid identifications to generate a plurality of protein groups.

In some embodiments, the performing the protein grouping comprises: (i) subdividing the harmonized mass spectrometry dataset to generate a plurality of mass spectrometry scans; (ii) distributing the plurality of mass spectrometry scans onto a plurality of computing nodes; and (iii) performing the plurality of polyamino acid searches, using the plurality of computing nodes, to generate the plurality of protein groups.

In some embodiments, each mass spectrometry scan in the plurality of mass spectrometry scans comprises a plurality of intensities for a plurality of retention times.

In some embodiments, the computer-implemented method further comprises (a) receiving a second mass spectrometry dataset; (b) generating a second event signal based on the mass spectrometry dataset; (c) instantiating a second serverless cloud computing instance based on the event signal; (d) performing a second data processing routine based on the second mass spectrometry dataset using the second serverless cloud computing instance, wherein the data processing routine and the second data processing routine are performed in parallel.

In some aspects, the present disclosure provides a computer-implemented method for processing a mass spectrometry (MS) dataset to store a trace in a distributed storage system: (a) extracting a plurality of signals from the MS dataset, wherein each signal in the plurality of signals comprises a mass-to-charge ratio (m/z), a retention time, and an intensity, wherein the plurality of signals is extracted when the m/z of a signal in the MS dataset is within a predetermined range from a reference m/z of a reference feature in the MS dataset; and (b) storing the trace comprising the plurality of signals in association with an identifier for the reference feature in the distributed storage system.

In some embodiments, the reference feature is annotated with a polyamino acid.

In some embodiments, the MS dataset comprises a columnar format.

In some embodiments, the computer-implemented method further comprises loading the MS dataset to a plurality of cache memories of a distributed computing system to generate a cached dataset.

In some embodiments, the computer-implemented method further comprises storing the cached dataset in the distributed storage system.

In some embodiments, the cached dataset is stored in a columnar format.

In some embodiments, the cached dataset is stored in a binary format.

In some embodiments, the computer-implemented method further comprises loading the cached dataset from the distributed storage system.

In some embodiments, the distributed storage system comprises an object-based storage system.

In some embodiments, the computer-implemented method further comprises loading the trace into a plurality of cache memories of a distributed computing system.

In some embodiments, the computer-implemented method further comprises displaying the trace on a graphical user interface.

In some embodiments, the computer-implemented method further comprises, before (a), identifying the reference feature in the MS dataset.

In some embodiments, the computer-implemented method further comprises, before (a), identifying a plurality of reference features in the MS dataset.

In some embodiments, the computer-implemented method further comprises extracting a second plurality of signals from the MS dataset based on a second reference feature in the MS dataset.

In some embodiments, the extracting the plurality of signals and the second plurality of signals is performed in parallel.

In some embodiments, the computer-implemented method further comprises storing a second trace comprising the second plurality of signals in association with a second identifier for the second reference feature in the distributed storage system.

In some embodiments, the storing the plurality of signals and the second plurality of signals is performed in parallel.

In some aspects, the present disclosure provides a method for identifying protein groups, comprising: (a) obtaining a plurality of independently measured mass spectrometry data; (b) subdividing each mass spectrometry data in the plurality of independently measured mass spectrometry data to provide a set of elements; (c) distributing the set of elements onto a plurality of nodes; and (d) generating, using the plurality of nodes, identifications of one or more biomolecules based at least in part on the set of elements.

In some embodiments, the plurality of independently measured mass spectrometry data comprises mass spectrometry data obtained by performing mass spectrometry on a plurality of biological samples.

In some embodiments, the plurality of nodes comprises a distributed computing system.

In some embodiments, the set of elements comprise a set of mass spectrometry scans.

In some embodiments, a first node in the plurality of nodes is configured to transfer one or more annotations in a first mass spectrometry scan to a second node in the plurality of nodes.

In some embodiments, the identifications comprise one or more peptide spectral matches.

In some embodiments, the set of elements comprise a set of peptide identifications.

In some embodiments, a first node in the plurality of nodes is configured to transfer one or more probability values associated with a protein group assignment for one or more peptide identifications in the set of peptide identifications to a second node in the plurality of nodes.

In some embodiments, the identifications comprise one or more protein group identifications.

In some aspects, the present disclosure provides a computer program product comprising a computer-readable medium having computer-executable code encoded therein, the computer-executable code adapted to be executed to implement any one of the computer-implemented methods disclosed herein.

In some aspects, the present disclosure provides a non-transitory computer-readable storage media encoded with a computer program including instructions executable by one or more processors to implement any one of the computer-implemented methods disclosed herein.

In some aspects, the present disclosure provides a computer-implemented system comprising: (a) a digital processing device comprising: (b) at least one processor, an operating system configured to perform executable instructions, a memory, and a computer program including instructions executable by the digital processing device to perform any one of the computer-implemented methods of claims disclosed herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C schematically illustrates a cloud scalable omics data analysis pipeline for processing MS datasets comprising a plurality of MS dataset filetypes, in accordance with some embodiments.

FIGS. 2A-2E schematically illustrate interfaces (i.e., an active programming interface (API), a graphical user interface (GUI), or both) for a cloud scalable omics data analysis pipeline, in accordance with some embodiments.

FIG. 3 shows a plot of total runtime as a function of the number of injections analyzed, in accordance with some embodiments.

FIG. 4 schematically illustrates a method for distributing a cached dataset and a task, in accordance with some embodiments.

FIG. 5 shows the computational costs for different processes in a label-free quantification analysis pipeline, in accordance with some embodiments.

FIGS. 6A-6B show the number of peptides identified using target-decoy and entrapment analysis, in accordance with some embodiments.

FIG. 7 schematically illustrates a process for performing alignment based on mass spectrometry datasets, in accordance with some embodiments.

FIG. 8 schematically illustrates a process for transmitting harmonized mass spectrometry datasets between computing nodes, in accordance with some embodiments.

FIG. 9 schematically illustrates a process for performing alignment based on harmonized mass spectrometry datasets, in accordance with some embodiments.

FIG. 10 schematically illustrates a computer system that is programmed or otherwise configured to implement methods provided herein.

FIG. 11 schematically illustrates a cloud-based distributed computing environment, in accordance with some embodiments.

FIG. 12 schematically illustrates a process for transmitting harmonized mass spectrometry datasets between computing nodes, in accordance with some embodiments.

### DETAILED DESCRIPTION

Though the human genome contains about 20,000 genes, some researchers estimate that the human proteome contains over 1 million proteins expressed from those genes. A number of different proteoforms can be expressed from a repertoire of various transcriptional, translational, and post-translational mechanisms (e.g., alternative splice forms, allelic variations, and protein modifications) that produce proteins that differ from those that comprise the canonical sequence expressed from the genes. Of the vast number of proteins estimated to exist in the human proteome, only a small fraction has thus been meaningfully identified and/or quantified in the human body.

Some of the challenges in identifying and quantifying the proteins is related to the rarity of certain proteins. For instance, human plasma contains protein species over a dynamic range that exceeds 12 magnitudes, where the top few proteins (e.g., albumin, transferrin, complement proteins, apolipoproteins, and alpha-2-macroglobulin) comprise 95% of the mass of protein in the plasma, and most of the protein species comprise the remaining 5%. Some of the protein species exist in the nanograms per milliliter ranges (e.g., transforming growth factor beta-1-induced transcript 1 protein at ˜10 ng/ml; fructose-bisphosphate aldolase A at ˜20 ng/ml; thioredoxin at ˜18 ng/ml; and L-selectin at ˜92 ng/ml), and some proteins are expected to present at level even beneath that range. Liquid chromatography coupled with mass spectrometry (LC-MS) or tandem mass spectrometry (LC-MS/MS) have grown into ubiquitous detection platforms due to their speed, sensitivity, and breadth of applications. LC-MS and LC-MS/MS can be used to identify protein species, however, due to the stochastic nature of the methods, only a fraction of ionic species that are generated at a time from a given sample may be selected for acquiring mass spectra. As a result, the presence of species that are highly abundant compared to the rare species can create an overwhelming amount of signals that make the rare species elusive.

Some aspects of the PROTEOGRAPH™ technology aims to solve some of these challenges by “compressing” the dynamic range of protein species in a sample. Some aspects of the PROTEOGRAPH™ technology operates based on non-specific binding of proteins to nanoparticle surfaces to form protein coronas. Without requiring a presence of a specific entity that is configured for binding to a singular specific protein (e.g., as in immunoassays), the non-specific binding can result in a dynamic range compression of proteins bound to the nanoparticle surfaces while capturing a wide variety of proteins. In other words, the relative abundance of proteins in the sample can be modified on the nanoparticle surfaces, such that the rare proteins are relatively more abundant, and the highly abundant proteins are relatively less abundant compared to the original sample. The proteins can then be separated from the sample and analyzed, for example, with mass spectrometry. The compressed dynamic range can allow rare proteins to comprise a higher fraction of ionic species, thereby allowing higher probability for detecting those rare proteins in a MS experiment. Though the above example is described in terms of proteins, other biomolecule classes (e.g., lipids, sugars, etc.) can be similarly targeted. Other aspects of the PROTEOGRAPH™ technology include controlled automation of the PROTEOGRAPH™ workflow that increases speed/throughput and accuracy/reliability.

While the introduction of the PROTEOGRAPH™ technology increased the number of proteins that can be detected from samples, another challenge is presented, which is to find biomarkers and/or therapeutic targets among those proteins. As the number of proteins that can be considered for diagnostic or therapeutic potential increases, the sample size may also be increased in order to effectively screen for the relevant proteins. Due to individual differences in biology between humans, thousands of proteins can have varying levels in plasma samples between two individuals. Therefore, samples from hundreds or thousands of individuals may be experimented with to identify meaningful and systematic signals that have clinical relevance.

Currently available platforms, software, and data structures used for processing mass spectrometry dataset have numerous limitations that make it difficult to process hundreds and thousands of samples. Some bioinformatic platforms use closed-source software and data structures, which make it difficult to cooperatively leverage mass spectrometry datasets across different users. For instance, some LC-MS and LC-MS/MS bioinformatic algorithms and software are built for desktop environments which are not easily leveraged for high-performance applications. Some LC-MS bioinformatic algorithms are closed-source “black-box” executables and cannot be distributed natively. Closed-source software can be difficult to leverage in distributed computing environments including cloud-based environments. Some software supporting a LC-MS instrument may output file formats that are different from another software supporting the LC-MS instrument. Dissonance between file formats obtained from different software or different mass spectrometry instruments can pose challenges in integrating data at scale. In some cases, differential proteomics data analysis of large datasets (‘group runs’) may require data aggregation (e.g., during chromatographic alignment or Protein Inference) of numerous and large datasets, which can be memory/disk limited in some environments, some existing applications are not designed for increasing compute and memory demands, and some software supporting a LC-MS instrument may not be designed optimally for computational speed or for efficiency in memory usage.

Improved computational platforms of the present disclosure can advantageously provide an ability to analyze mass spectrometry datasets from hundreds, thousands, or more mass spectrometry experiments. Some of the challenges addressed by the systems and methods of the present disclosure include harmonizing a large variety of mass spectrometry dataset formats so that the datasets can be processed together. Another aspect includes providing a number of mass spectrometry analysis algorithms on a singular platform. The harmonization employed by the computational platforms of the present disclosure can allow users of the platform to utilize mass spectrometry datasets from disparate sources (e.g., datasets from different machines, different locations, different times, etc.) using a variety of mass spectrometry analysis algorithms (some current algorithms may require a specific type of a dataset format—by harmonizing the datasets, algorithms can be used a harmonized dataset regardless of the source). The modularization can allow users of the platform to write new programs and computational protocols for processing or analyzing mass spectrometry datasets using the variety of mass spectrometry analysis algorithm. The computational platforms of the present disclosure can provide remote access to multiple users and entities over a network. Datasets can be shared between remote users in real-time in harmonized formats, regardless of the format that the datasets were originally generated by the users. The following paragraphs provide illustrative embodiments that detail various aspects of the computational platforms of the present disclosure.

### Storing and Processing Mass Spectrometry Datasets

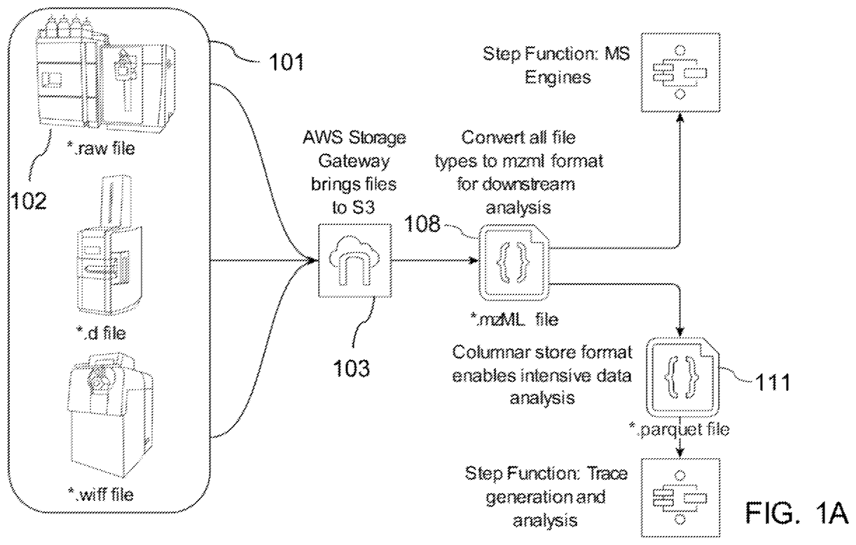

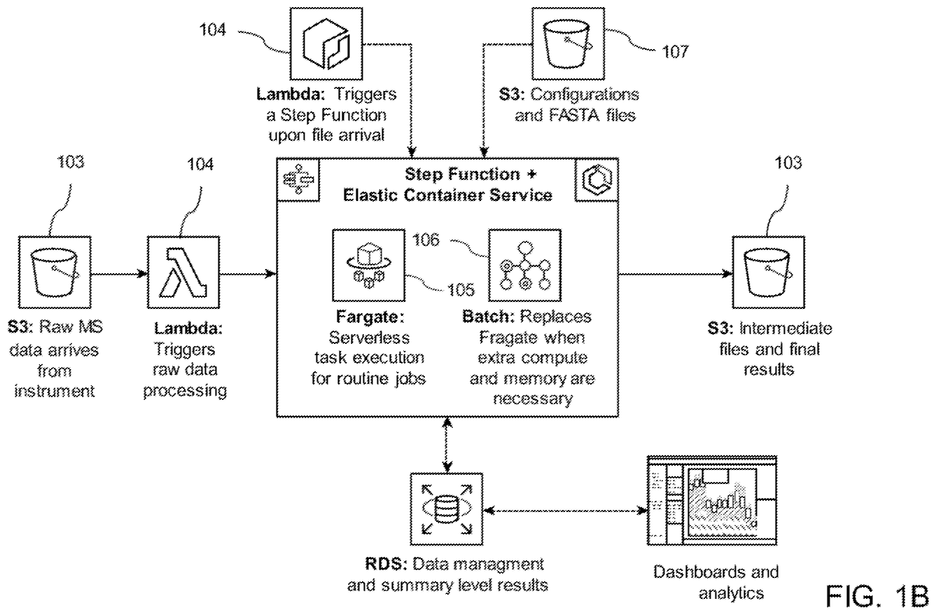

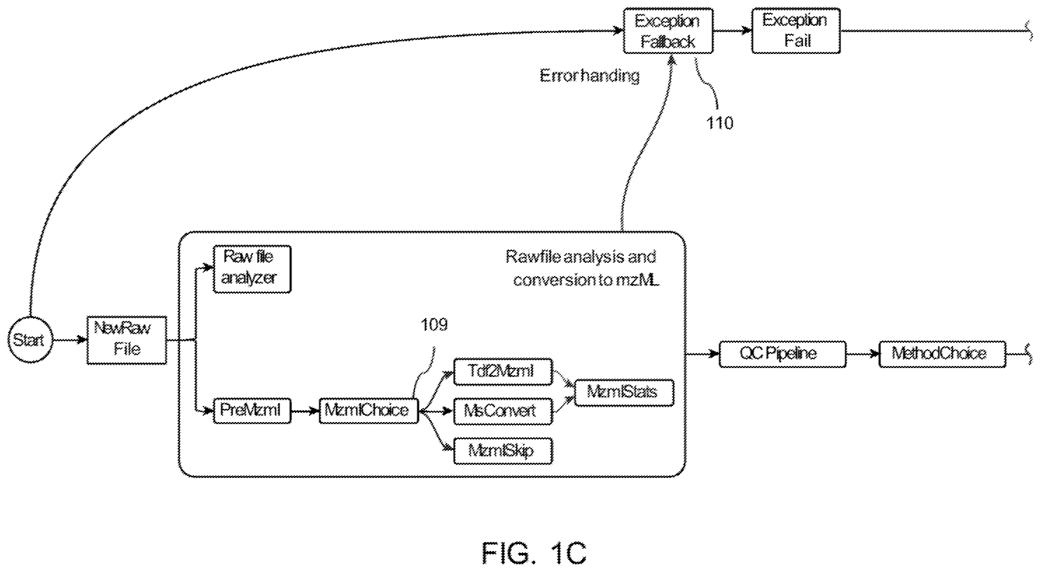

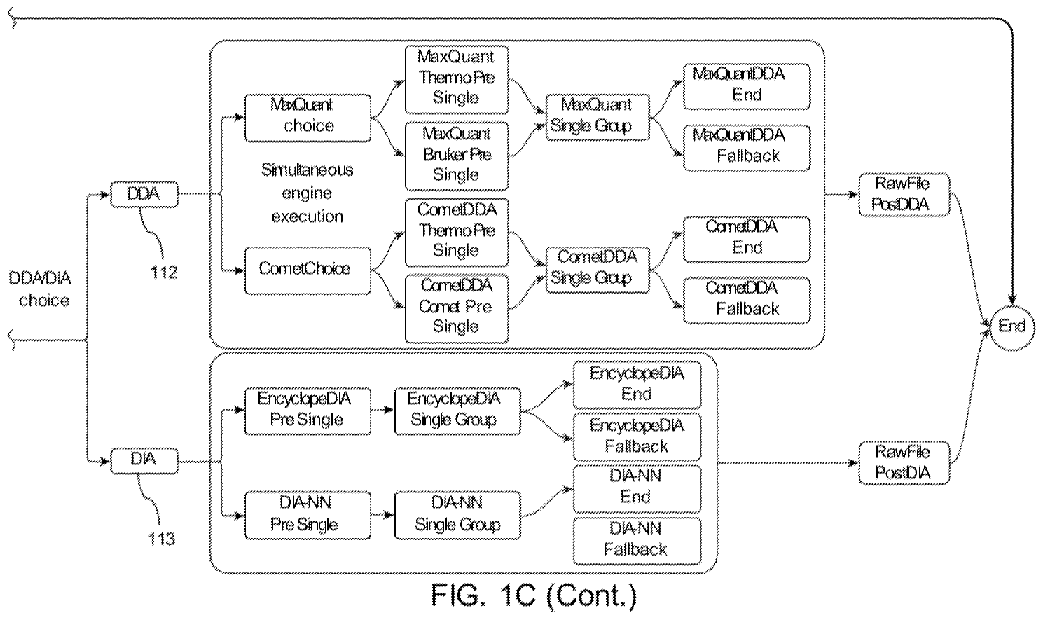

In some aspects, the present disclosure provides a computer-implemented method for storing and processing mass spectrometry datasets on a cloud platform. FIGS. 1A-1B schematically illustrate a cloud scalable mass spectrometry data analysis pipeline for processing outputs from a plurality of mass spectrometry (MS) instrument types, in accordance with some embodiments. The computer-implemented method can comprise transmitting a mass spectrometry dataset (101) to a computer system. The transmitting can be performed autonomously. The computer-implemented method can comprise receiving the mass spectrometry dataset at the computer system. The computer-implemented method can comprise transmitting a plurality of mass spectrometry datasets to the computer system. The computer-implemented method can comprise receiving the plurality of mass spectrometry datasets at the computer system.

The mass spectrometry dataset can be generated by a mass spectrometer (102). The mass spectrometry dataset can be generated by a plurality of mass spectrometers. The mass spectrometer can transmit the mass spectrometry dataset autonomously. The mass spectrometry dataset can comprise data from a set of experiments, a set of measurements (e.g., data from one or more injections in a tandem liquid chromatography-mass spectrometry experiment) in a single experiment, or both. The mass spectrometry dataset can be accompanied by a user-specified recipes or settings for processing the mass spectrometry dataset. The plurality of mass spectrometers can be at different locations. The plurality of mass spectrometers can generate the mass spectrometry datasets during the same time period or at different time periods from one another. The plurality of mass spectrometers may be operated by the same entity or different entities (e.g., customers, users, companies, labs, researchers, etc.). The mass spectrometer can comprise a plurality of mass spectrometer types or commercial models. The plurality of mass spectrometer types or commercial models can generate a plurality mass spectrometry datasets comprising a variety of data formats. The mass spectrometry dataset can comprise one of a plurality of mass spectrometry dataset formats. Mass spectrometry dataset formats can include *.raw format, *.d format, *.wiff format, *.txt format, or any other format used for storing or processing mass spectrometry data. The mass spectrometry dataset can be stored on a cloud-based storage system (103).

Upon receiving the mass spectrometry dataset, an event signal can be generated by the computer system. The event signal can be configured to trigger an event on the computer system. The event signal can be used as a trigger to create a serverless cloud computing instance for running a data processing routine. The event signal can be used as a trigger to create a container for running a data processing routine. The event signal can be used to trigger (104) the data processing routine to be performed on the mass spectrometry dataset using the serverless cloud computing instance (105). If the a serverless cloud computing instance cannot be instantiated (e.g., when resources for serverless cloud computing are limited), the data processing routine can be performed using a server cloud computing instance (106). The size of computational resources of the serverless cloud computing instance can be based on the mass spectrometry dataset. For instance, the size of the computational resources can be scaled autonomously based on the size and/or complexity of the mass spectrometry dataset. A computational resource can comprise memory, storage, number of processors, or any combination thereof. The computer-implemented method can comprise receiving a second mass spectrometry dataset. A second event signal can be generated based on the second mass spectrometry dataset. A second serverless cloud computing instance can be created based on the second event signal. A second data processing routine can be performed based on the second mass spectrometry dataset using the second serverless cloud computing instance. The data processing routine and the second data processing routine can be performed in parallel. In some embodiments, the computer-implemented method can process and/or store genomic datasets (107) on the cloud platform. For each new mass spectrometry dataset that is received, a new serverless cloud computing instance can be instantiated to perform the data processing routine on each mass spectrometry dataset.

The data processing routine can comprise generating a harmonized mass spectrometry dataset (108) comprising a harmonized data format based on the mass spectrometry dataset. A harmonized mass spectrometry dataset can refer to a mass spectrometry dataset that has a been transformed to have a consistent format with another mass spectrometry dataset. The harmonized mass spectrometry dataset can be an *.xml, *.h5, *.mzml, *.parquet, or any appropriate format. The harmonized mass spectrometry dataset can comprise headers, sections, indices, columns, rows, graphs and any other organizational structure for organizing MS data. An example of a data processing routine is schematically illustrated in FIG. 1C. The data processing routine can receive a MS dataset. Depending on the format of the MS dataset, different conversion algorithms (109) can be used to generate the harmonized MS dataset. The data processing routine can comprise error and/or exception handling routines (110). The error and/or exception handling routines can notify an entity (e.g., a user) of an error. The error and/or exception handling routines can provide suggestions for troubleshooting or solving the error. The data processing routine can comprise generating a plurality of harmonized mass spectrometry datasets comprising the harmonized data format based on a plurality of mass spectrometry datasets. In some embodiments, the harmonized mass spectrometry dataset comprises a columnar format (111), e.g., *.parquet format. The data processing routine can comprise storing the harmonized mass spectrometry dataset on storage system. The storage system can be an object-based storage system. The object-based storage system can be partitioned to create space for storing the harmonized mass spectrometry dataset. The space can be autonomously scaled based on the size of the harmonized mass spectrometry dataset. The data processing routine can comprise processing the harmonized mass spectrometry dataset after retrieving it from the storage system.

The data processing routine can comprise performing a polyamino acid search to generate a plurality of polyamino acid identifications. Polyamino acid can refer to a peptide, a protein, or any molecule or complex comprising two or more amino acids in a sequence. A polymino acid search can refer to a process for determining an identity (e.g., a sequence, a protein group, an isoform in a protein group, etc.) of a polyamino acid based on information about the polyamino acid. The data processing routine can comprise performing a plurality of polyamino acid searches. The polyamino acid search can be based on the harmonized mass spectrometry dataset and a data acquisition mode of the mass spectrometry dataset. The data acquisition mode of the mass spectrometry dataset can be data dependent acquisition (DDA) or data independent acquisition (DIA). The polyamino acid search can be one or more of a plurality of search modes. The plurality of search modes can comprise a plurality of DDA search modes (112) or a plurality of DIA (113) search modes. For instance, a DDA search mode can be MaxQuant, CometDDA, or another search mode configured to process DDA datasets. A DIA search mode can be EncylopeDIA, DIA-NN, or another search mode configured to process DIA datasets. The data processing routine can comprise storing the plurality of polyamino acid identifications on the storage system. The storage system can be an object-based storage system. The storage system can be a distributed relational storage system. The storage system can be a non-relational storage system. The storage system can be a public storage system, a shared storage system between two or more entities, or a private storage system.

The data processing routine can comprise performing protein grouping based on the plurality of polyamino acid identifications to generate a plurality of protein groups. Performing the protein grouping can comprise subdividing the harmonized mass spectrometry dataset to generate a plurality of mass spectrometry scans. Performing the protein grouping can comprise distributing the plurality of mass spectrometry scans onto a plurality of computing nodes. Performing the protein grouping can comprise performing the plurality of polyamino acid searches, using the plurality of computing nodes, to generate the plurality of protein groups. The data processing routine can comprise normalizing the mass spectrometry dataset. The data processing routine can comprise alignment, quantification, or both.

In some embodiments, the computer-implemented method comprises processing a mass spectrometry (MS) dataset to store a trace in a distributed storage system. The computer-implemented method can comprise extracting a plurality of signals from the MS dataset. Each signal in the plurality of signals can comprise a mass-to-charge ratio (m/z), a retention time, and an intensity. The plurality of signals can be extracted when the m/z of a signal in the MS dataset is within a predetermined range from a reference m/z of a reference feature in the MS dataset. The trace comprising the plurality of signals in association with an identifier for the reference feature can be stored in the distributed storage system. The trace can be loaded into a cache memory for further processing, for example, visualizing the trace, determining a quality of the trace, quantifying the statistics of the trace, and etc.

In some aspects, the present disclosure provides a computer-implemented system for storing mass spectrometry datasets on a cloud platform. The computer-implemented system can comprise at least one digital processing device. The at least one digital processing device can comprise at least one processor, an operating system configured to perform executable instructions, a memory, and a computer program including instructions executable by the digital processing device. The instructions can comprise a first instruction configured to generate an event signal when a mass spectrometry dataset is received by the computer-implemented system. The mass spectrometry dataset can comprise at least one of a plurality of formats. The instructions can comprise a second instruction configured to be triggered by the event signal to instantiate a serverless cloud computing instance. The instructions can comprise a third instruction configured to perform a data processing routine using the serverless cloud computing instance. The data processing routine can comprise generating a harmonized mass spectrometry dataset comprising a harmonized data format based on the mass spectrometry dataset. The data processing routine can comprise storing the harmonized mass spectrometry dataset on an object-based storage system.

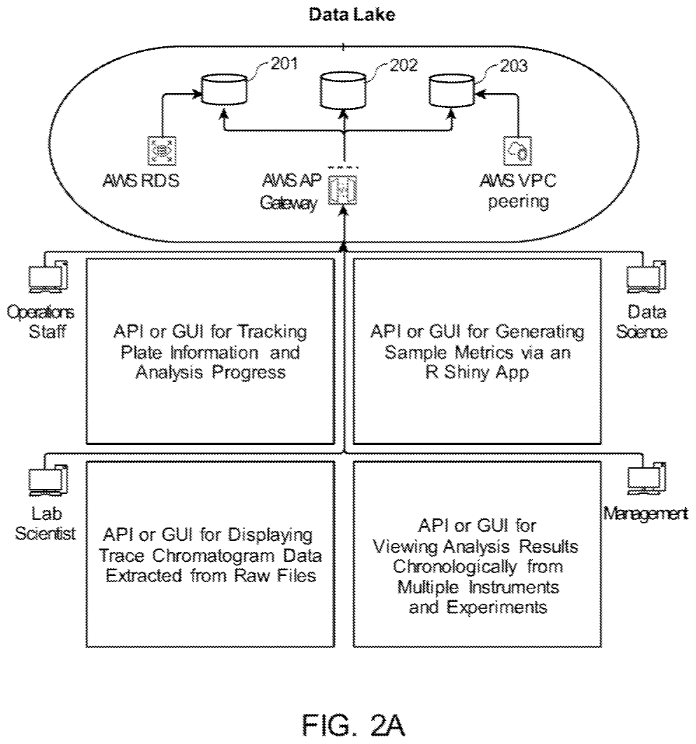

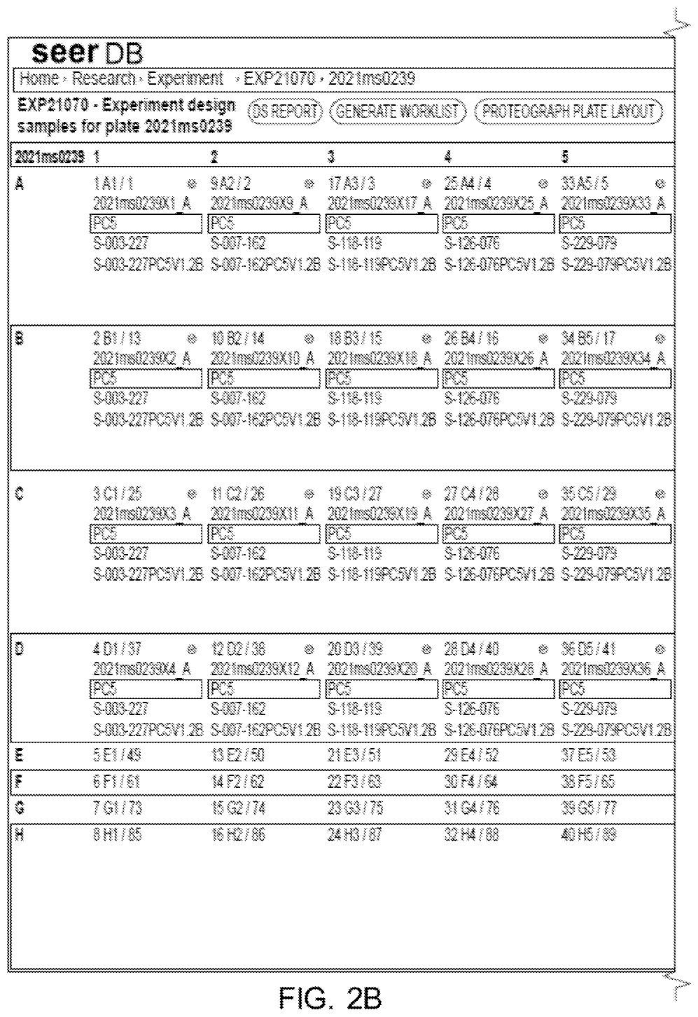

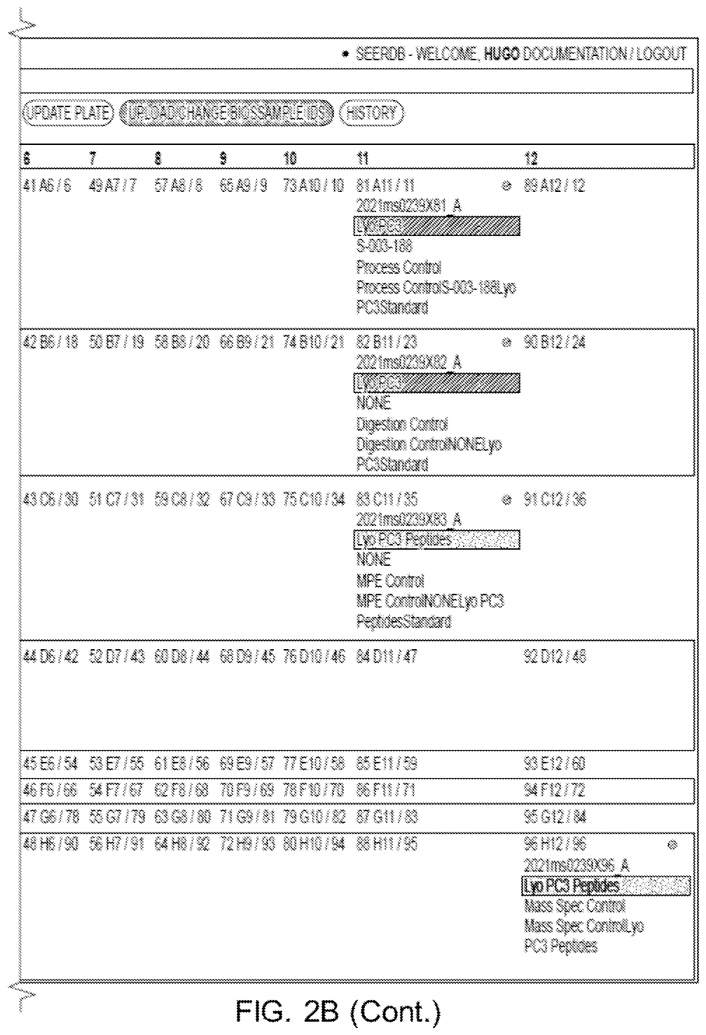

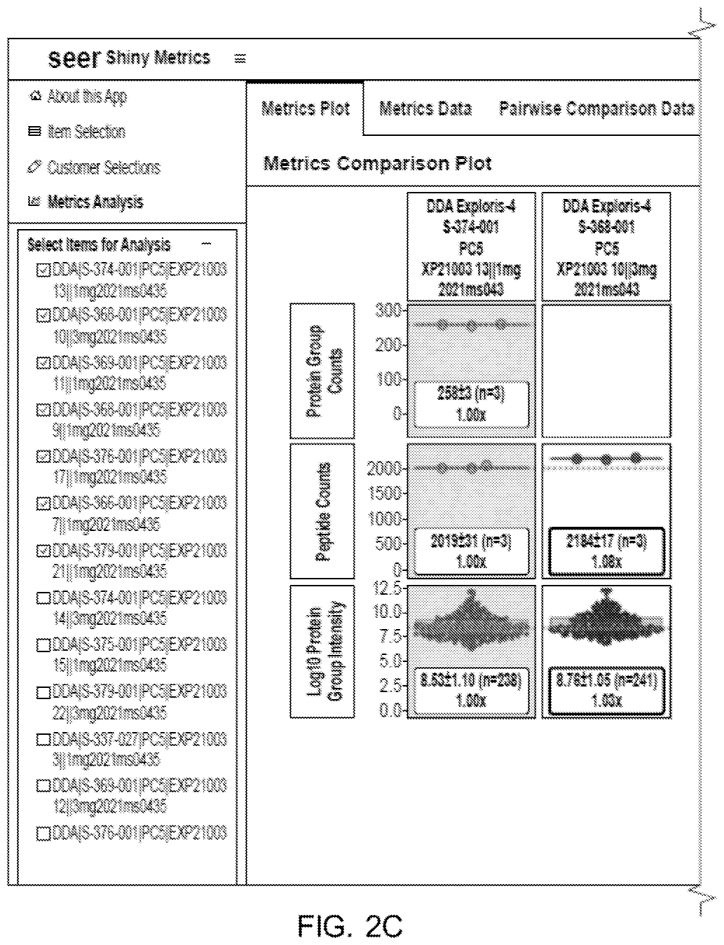

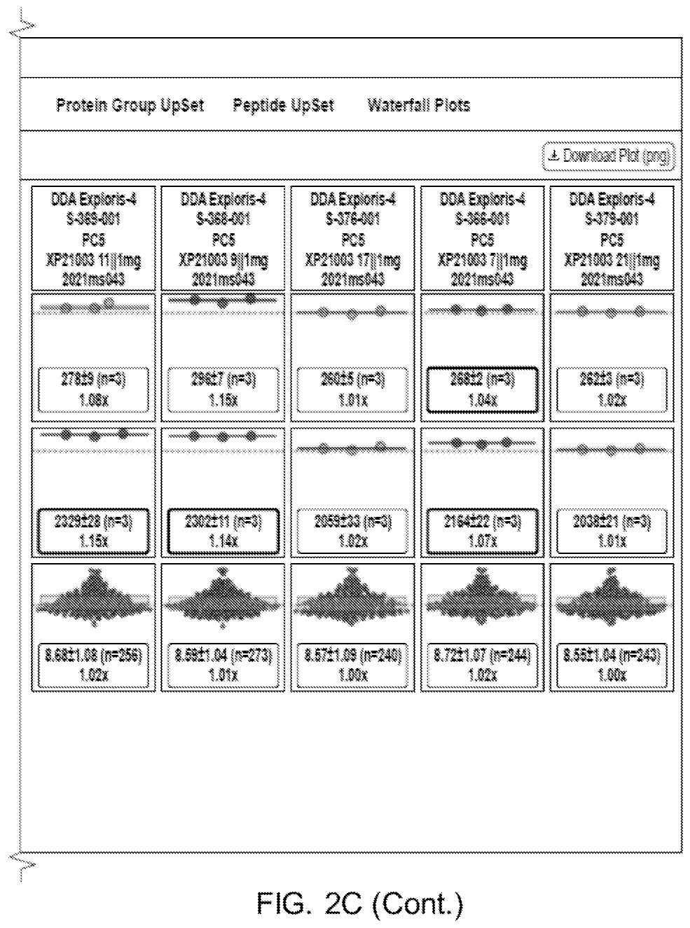

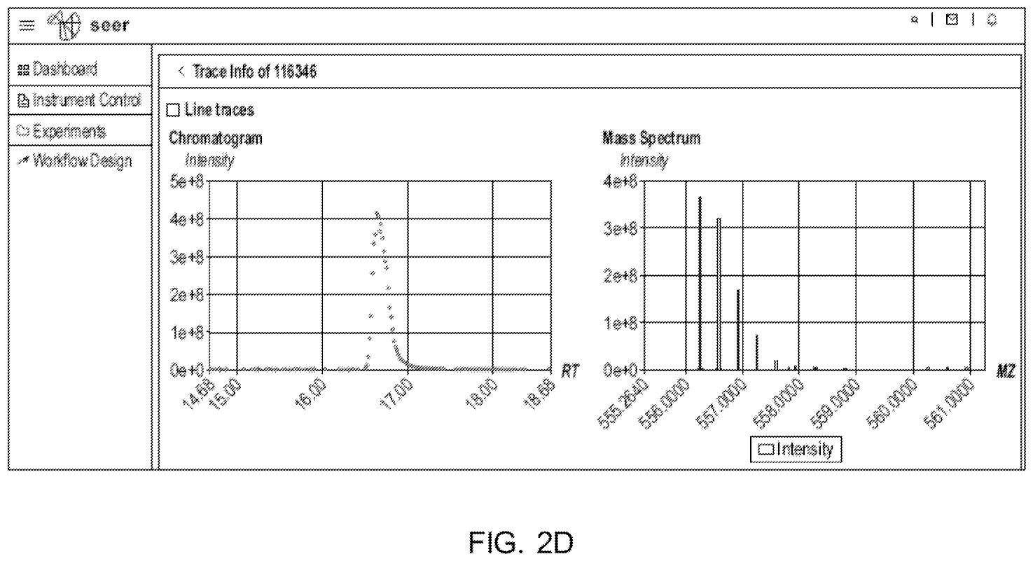

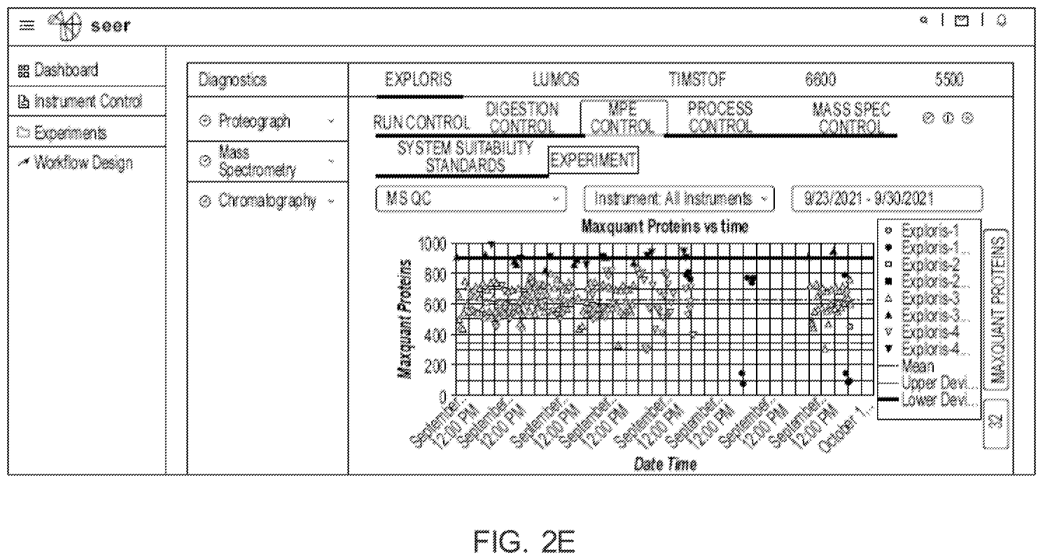

The computer-implemented system can comprise one or more databases. A database can be a distributed relational database (201). A database can be an object-based distributed database (202). A database can be on a server. A database can be a non-relational database (203). A database can be public database, a shared database between two or more entities, or a private database only accessible by one entity. The computer-implemented system can comprise an application programming interface (API) or a GUI. FIGS. 2A-2E schematically illustrates an GUI for a cloud scalable omics data analysis pipeline, in accordance with some embodiments. An API or GUI can track the progress of experiments (e.g., plate information) and data processing routines. For instance, FIG. 2B schematically illustrates a GUI for tracking plate information and analysis for an experiment, in accordance with some embodiments. An API or GUI can be used to generate or visualize metrics for experiments and data processing routines. FIG. 2C schematically illustrates a GUI for generating sample metrics for an experiment, in accordance with some embodiments. An API or GUI can be used to generate or visualize traces. FIG. 2D schematically illustrates a GUI for displaying a trace of an MS feature extracted from a MS dataset from an experiment, in accordance with some embodiments. An API or GUI can be used to generate or visualize metrics for experiment results from multiple instruments, experiments, or both. FIG. 2E schematically illustrates a GUI for viewing analysis results chronologically from multiple experiments conducted on multiple instruments, in accordance with some embodiments. The API or the GUI can be programmed de novo, reprogrammed, or reconfigured by a user to perform new functions.

In some embodiments, the processing further comprises identifying a biomarker in the plurality of harmonized mass spectrometry datasets. In some embodiments, the plurality of harmonized mass spectrometry datasets are differential in at least one clinically relevant dimension. In some embodiments, the biomarker is associated with the at least one clinically relevant dimension. In some embodiments, the processing further comprises performing a power curve analysis based on the plurality of harmonized mass spectrometry datasets. In some embodiments, the power curve analysis provides a statistical power for identifying a biomarker based on the plurality of harmonized mass spectrometry datasets. In some embodiments the power curve analysis provides a ratio between a number of samples to a number of potential biomarkers that can be found with a predetermined statistical significance value. In some embodiments, the processing further comprises training a machine learning model based on the plurality of harmonized mass spectrometry datasets. In some embodiments, the processing further comprises performing clustering analysis based on the plurality of harmonized mass spectrometry datasets. The biomarker can comprise a level of a signal for a biomolecule in a subset in a fraction of the plurality of harmonized mass spectrometry datasets. The biomarker can comprise levels for a plurality of signals for a plurality of biomolecules in a subset in a fraction of the plurality of harmonized mass spectrometry datasets.

### Normalizing Mass Spectrometry Datasets

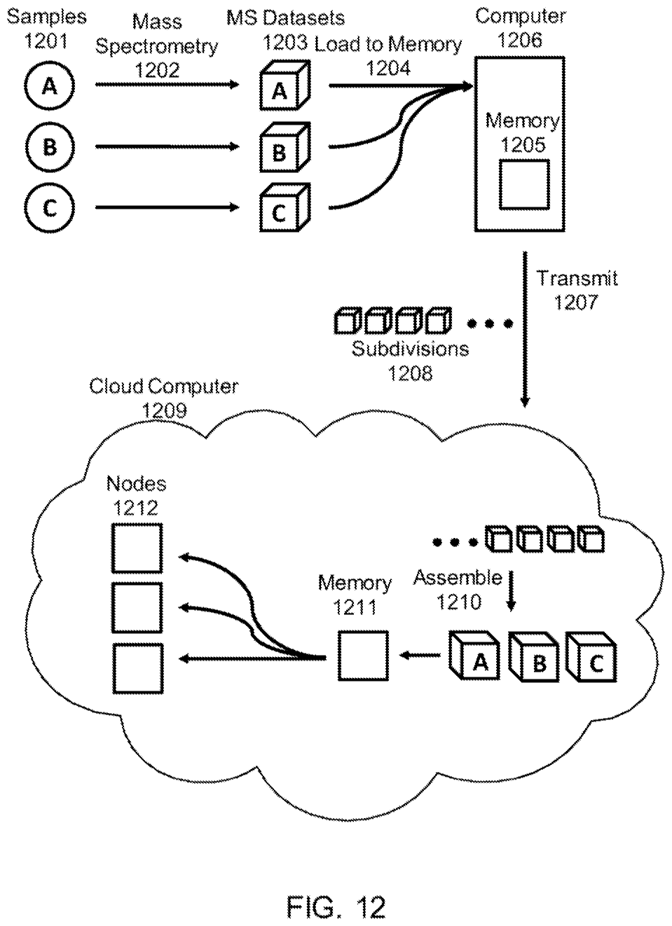

In some aspects, the present disclosure provides a computer-implemented method for normalizing and processing mass spectrometry datasets. FIG. 12 schematically illustrates a computer-implemented method for transmitting harmonized mass spectrometry datasets between computing nodes, in accordance with some embodiments. The computer-implemented method can comprise obtaining a plurality of mass spectrometry datasets (1203) obtained from a plurality of samples (1201). The plurality of mass spectrometry datasets can be obtained by performing mass spectrometry (1202) on the plurality of samples. The plurality of mass spectrometry datasets can comprise a plurality of harmonized mass spectrometry datasets. In some embodiments, the harmonized dataset are obtained through the method of storing and processing mass spectrometry datasets discussed above. For example, mass spectrometry datasets are converted to a plurality of harmonized mass spectrometry datasets as depicted FIG. 1A. In some embodiments, the computer-implemented method comprises loading (1204) the plurality of mass spectrometry datasets into a memory (1205) of a computing node (1206) to generate a cached dataset. The computer-implemented method can comprise transmitting (1207) a copy of the cached dataset (1208) to a plurality of cache memories of a plurality of computing nodes (1212). The transmitting can be performed using one or more of a variety of wired and/or wireless connections. In some embodiments, the computer-implemented method comprises determining, using the plurality of computing nodes, a plurality of feature values for the plurality of mass spectrometry datasets. The computer-implemented method can comprise normalizing, using the plurality of computing nodes, across the plurality of mass spectrometry datasets using the plurality of feature values to generate a plurality of normalized mass spectrometry datasets. In some embodiments, the computer-implemented method comprises processing the plurality of normalized mass spectrometry datasets to compare the plurality of samples.

In some embodiments, the plurality of mass spectrometry datasets (1203) comprises a set of precursors for each sample in the plurality of samples. In some embodiments, the set of precursors comprises a set of biomolecule precursors. In some embodiments, the set of biomolecule precursors comprises a set of polyamino acid precursors.

In some embodiments, the plurality of mass spectrometry datasets (1203) comprises information about a single cell, a tissue, an organ, a system of tissues and/or organs (such as cardiovascular, respiratory, digestive, or nervous systems), or an entire multicellular organism. In some embodiments, the plurality of mass spectrometry datasets comprises information about an individual (e.g., an individual human being or an individual bacterium), or a population of individuals (e.g., human beings with diagnosed with cancer or a colony of bacteria). The plurality of mass spectrometry datasets may comprise information from various forms of life, including forms of life from the Archaca, the Bacteria, the Eukarya, the Protozoa, the Chromista, the Plantae, the Fungi, or from the Animalia. In some embodiments, the plurality of mass spectrometry datasets may comprise information from viruses.

In some embodiments, the plurality of mass spectrometry datasets (1203) comprises a set of chemical identifications for each sample in the plurality of samples. In some embodiments, the set of chemical identifications comprises a set of biomolecule identifications. In some embodiments, the set of biomolecule identifications comprises a set of polyamino acid identifications. In some embodiments, the set of polyamino acid identifications comprises a set of tryptic or semi-tryptic peptide identifications. In some embodiments, the plurality of mass spectrometry datasets comprises a set of chemical intensities for each sample in the plurality of samples. In some embodiments, the set of chemical intensities comprises a set of biomolecule intensities. In some embodiments, the set of biomolecule intensities comprises a set of polyamino acid intensities. In some embodiments, the set of polyamino acid intensities comprises a set of tryptic or semi-tryptic peptide intensities. In some embodiments, the set of polyamino acid identifications comprises a set of protein group identifications. In some embodiments, the set of polyamino acid intensities comprises a set of protein group intensities.

In some embodiments, the plurality of mass spectrometry datasets (1203) comprises a data independent acquisition (DIA) mass spectrometry dataset, a data dependent acquisition (DDA) mass spectrometry dataset, or both. In some embodiments, the plurality of mass spectrometry datasets comprises a LC-MS dataset, a LC-MS/MS dataset, or both. The mass spectrometry (1202) can comprise a LC-MS dataset, a LC-MS/MS dataset, or both. The mass spectrometry can be performed with DIA, DDA, or both.

As discussed further below, the plurality of mass spectrometry datasets (1203) may be derived, for example, from biological samples (e.g., plasma, etc.). In addition, the plurality of mass spectrometry datasets (1203) may be derived, for example, from samples where biomolecules, such as peptides or proteins, have been selectively enriched. In addition, the plurality of mass spectrometry datasets (1203) may be derived, for example, from samples where non-specific binding to surfaces (e.g., to two or more different nanoparticles have different physicochemical properties) has been used to compress the dynamic range of the sample.

In some embodiments, the computing node (1206) is a local computing node. In some embodiments, the local computing node comprises a computing device interfacing with a user. In some embodiments, a desktop computer, a laptop computer, or a mobile device comprises the local computing node. In some embodiments, an instrument comprises the local computing node. In some embodiments, a mass spectrometry or a sequencing instrument comprises the local computing node. In some embodiments, the computing node comprises a cloud-computing node.

In some embodiments, the plurality of computing nodes (1212) comprises a plurality of cloud-computing nodes. In some embodiments, a cloud-computing cluster comprises one or more cloud-computing nodes. In some embodiments, an instance comprises one or more cloud-computing clusters. In some embodiments, a plurality of computing nodes comprises the computing node. In some embodiments, the plurality of computing nodes comprises at least 2, 5, 10, 100, 1000, 10000, or 100000 computing nodes. In some embodiments, the plurality of computing nodes comprises at most 10, 100, 1000, 10000, 100000, or 1000000 computing nodes. In some embodiments, a cloud computing node comprises a virtual machine instance. The number of nodes in the plurality of nodes can be autonomously scaled based on the size or amount of the mass spectrometry datasets, the complexity of the task to be performed using the mass spectrometry datasets, or both.

In some embodiments, the memory (1205) comprises a random access memory (RAM). In some embodiments, the memory comprises a cache memory. In some embodiments, the cache memory may comprise a level 1, level 2, level 3, level 4 cache memory, or any combination thereof. In some embodiments, the cache memory may comprise at least 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB. In some embodiments, the cache memory may comprise at most 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB. In some embodiments, a plurality of cache memories comprises the cache memory. In some embodiments, a plurality of computing nodes may comprise the plurality of cache memories. In some embodiments, the plurality of cache memories can be in operable communication with a plurality of buses for transmitting or receiving data. The transmitting or receiving can be performed using one or more of a variety of wired and/or wireless connections. The plurality of buses can comprise various protocols and technologies, including Modem, LTE, GSM, DOCSIS, OC, Ethernet, Infiniband, IEEE 802.11, Bluetooth, for example. The plurality of buses can comprise a bit rate of at least 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB per second. The plurality of buses can comprise a bit rate of at most 32 kilobytes (KB), 64 KB, 128 KB, 256 KB, 512 KB, 1 megabyte (MB), 2 MB, 4 MB, 8 MB, 16 MB, 32 MB, 64 MB, 128 MB, 256 MB, 512 MB, 1 gigabyte (GB), 2 GB, 4 GB, 8 GB, 16 GB, 32 GB, 64 GB, 128 GB, 256 GB, or 512 GB per second.

In some embodiments, the cached dataset is an unserialized cached dataset. In some embodiments, the unserialized cached dataset is serialized to generate a serialized cached dataset. In some embodiments, the serialized cached dataset comprises a series of bytes. In some embodiments, the serialized cached dataset is subdivided to generate a subdivided cached dataset. In some embodiments, the subdivided cached dataset may comprise a plurality of subdivisions. In some embodiments, a subdivision may comprise at least 8 bytes (B), 16 B, 32 B, 64 B, 128 B, 256 B, 512 B, 1 KB, 2 KB, 4 KB, 8 KB, 16 kB, 32 kB, 64 KB, 128 kB, 256 kB, 512 KB, 1 MB, 2 MB, 4 MB, 8 MB, 16 MB, 32 MB, 64 MB, 128 MB, 256 MB, 512 MB, or 1 GB.

In some embodiments, the transmitting (1207) comprises transmitting the plurality of subdivisions of the subdivided cached dataset. In some embodiments, the plurality of subdivisions are transmitted one subdivision at a time. In some embodiments, the plurality of subdivisions are transmitted more than one subdivision at a time. In some embodiments, the transmitting comprises assembling a copy of the serialized cached dataset from the copy of the subdivided cache. In some embodiments, the copy of the serialized cached dataset is assembled at a computing node in the plurality of computing nodes.

The plurality of mass spectrometry datasets (1203) can be a plurality of harmonized mass spectrometry datasets. The plurality of mass spectrometry datasets can comprise a columnar format. The plurality of mass spectrometry datasets can be stored on a distributed storage system. The plurality of mass spectrometry datasets can be stored on an object-based storage system. The plurality of mass spectrometry datasets can be stored on a distributed relational storage system. The plurality of mass spectrometry datasets can be stored on a non-relational storage system. The plurality of mass spectrometry datasets can be stored on a public storage system, a shared storage system between two or more entities, or a private storage system.

The amount of time that it takes to process a mass spectrometry dataset can be significantly reduced. In some embodiments, a processing time for one or more processes of the computer-implemented method may be substantially linear as a function of a number of mass spectrometry datasets in the plurality of mass spectrometry datasets. In some embodiments, performing for one or more processes of the computer-implemented method may take less than ax^{1.5}, ax^{1.6}, ax^{1.4}, or ax^{1.2} amount of compute time, wherein x is a number of mass spectrometry datasets in the plurality of mass spectrometry datasets, and wherein a is a constant. In some embodiments, performing for one or more processes of the computer-implemented method may take less than ax^{1.8}, ax^{1.6}, ax^{1.4}, or ax^{1.2} amount of real time, wherein x is a number of mass spectrometry datasets in the plurality of mass spectrometry datasets, and wherein a is a constant.

In some embodiments, the processing further comprises determining a biomarker in the plurality of mass spectrometry datasets. In some embodiments, the processing further comprises determining a biomarker based on the plurality of normalized mass spectrometry datasets. In some embodiments, the plurality of samples are differential in at least one clinically relevant dimension. In some embodiments, the biomarker is associated with the at least one clinically relevant dimension. In some embodiments, the processing further comprises performing a power curve analysis based on the plurality of normalized mass spectrometry datasets. In some embodiments, the power curve analysis provides a statistical power for identifying a biomarker based on the plurality of normalized mass spectrometry datasets. In some embodiments the power curve analysis provides a ratio between a number of samples to a number of potential biomarkers that can be found with a predetermined statistical significance value. In some embodiments, the processing further comprises training a machine learning model based on the plurality of normalized mass spectrometry datasets. In some embodiments, the processing further comprises performing clustering analysis based on the plurality of normalized mass spectrometry datasets. The biomarker can comprise a level of a signal for a biomolecule in a subset in a fraction of the plurality of mass spectrometry datasets. The biomarker can comprise levels for a plurality of signals for a plurality of biomolecules in a subset in a fraction of the plurality of mass spectrometry datasets.

### Alignment

In some embodiments, a method of the present disclosure may comprise normalizing, using a plurality of computing nodes, across a plurality of mass spectrometry datasets using a plurality of feature values to generate a plurality of normalized mass spectrometry datasets. In some embodiments, the plurality of mass spectrometry datasets may be normalized such that a chemical identification from one mass spectrometry dataset in the plurality of mass spectrometry datasets may be used to identify another chemical in another mass spectrometry dataset in the plurality of mass spectrometry datasets. In some embodiments, a feature value may be applied to a mass spectrometry dataset in a relative fashion (i.e., applied to mass-to-charge ratio and mobility) or in an absolute fashion (i.e., applied to retention time).

In some embodiments, the aligning may be based on a plurality of feature values. In some embodiments, the plurality of feature values comprises a feature value for the set of precursors of each mass spectrometry dataset in the plurality of mass spectrometry datasets. In some embodiments, the feature value is configured for normalizing retention time, mass-to-charge ratio, ion mobility, or a combination thereof. In some embodiments, the feature value is a shifting value. In some embodiments, the shifting value is added to the retention time, mass-to-charge ratio, or ion mobility for a mass spectrometry dataset in the plurality of mass spectrometry datasets.

In some embodiments, the feature values are based on isotopic clusters. In some embodiments, the feature values comprise retention time, mass-to-charge ratio, aggregate peak area of the isotope cluster, ion mobility, or any combination thereof. In some embodiments, the normalizing generates a set of aligned precursors for each mass spectrometry dataset in the plurality of mass spectrometry datasets. In some embodiments, the normalizing further comprises identifying a first chemical from a first mass spectrometry dataset in the plurality of mass spectrometry datasets based on an aligned precursor in the set of aligned precursors of a second mass spectrometry dataset.

In some embodiments, the determining comprises minimizing an objective function, using a computing node in the plurality of computing nodes, based on a pair of mass spectrometry datasets in the plurality of mass spectrometry datasets. In some embodiments, the determining comprises minimizing the objective function for a unique pair of mass spectrometry datasets in the plurality of mass spectrometry datasets for each computing node in the plurality of computing nodes.

### Quantification

In some embodiments, a method of the present disclosure may comprise normalizing, using a plurality of computing nodes, across a plurality of mass spectrometry datasets using a plurality of feature values to generate a plurality of normalized mass spectrometry datasets. In some embodiments, the normalizing may be performed to determine intensities of chemicals in the plurality of mass spectrometry datasets. In some embodiments, the intensities of chemicals may be determined such that comparisons can be made between individual mass spectrometry datasets in the plurality of mass spectrometry datasets. In some embodiments, the normalizing comprises label-free quantification. In some embodiments, the normalizing generates a set of relative abundances for each mass spectrometry dataset in the plurality of mass spectrometry datasets.

In some embodiments, a feature value in the plurality of feature values may be determined by minimizing an objective function, using a computing node in the plurality of computing nodes, based on a pair of mass spectrometry datasets in the plurality of mass spectrometry datasets. In some embodiments, the objective function is minimized for a unique pair of mass spectrometry datasets in the plurality of mass spectrometry datasets for each computing node in the plurality of computing nodes.

In some embodiments, the objective function comprises:

$$L = {\sum }_{p}^{N} \vert \frac{I ( {Norm}_{A} , p )}{I ( {Norm}_{B} , p )} \vert ,$$

wherein N is a number of chemical identifications in the set of chemical identifications, wherein p is a chemical in the set of chemical identifications, wherein I is an intensity value for the set of chemical intensities, wherein Norm_{A} is a first feature value for a first mass spectrometry dataset in the pair of mass spectrometry datasets, and wherein Norm_{B} is a second feature value for a second mass spectrometry dataset in the pair of mass spectrometry datasets.

In some embodiments, the objective function comprises:

$$L = {\sum }_{A , B}^{M} {\sum }_{p}^{N} \vert \frac{I ( {Norm}_{A} , p , A )}{I ( {Norm}_{B} , p , B )} \vert ,$$

wherein M is a number of unique pairs of mass spectrometry datasets in the plurality of mass spectrometry datasets, and wherein A,B is the unique pair of mass spectrometry datasets in the plurality of mass spectrometry datasets.

In some embodiments, the set of relative abundances comprises a set of chemical relative abundances. In some embodiments, the set of chemical relative abundances comprises a set of biomolecule relative abundances. In some embodiments, the set of biomolecule relative abundances comprises a set of polyamino acid relative abundances. In some embodiments, the set of relative abundances represent relative abundances of chemicals between the plurality of mass spectrometry datasets. In some embodiments, the set of relative abundances represent relative abundances of polyamino acids between the plurality of mass spectrometry datasets. In some embodiments, the plurality of feature values comprises a feature value for the set of chemical intensities of each mass spectrometry dataset in the plurality of mass spectrometry datasets. In some embodiments, the normalizing comprises adjusting the set of chemical intensities for each mass spectrometry dataset in the plurality of mass spectrometry datasets based on the plurality of feature values.

### Identification

In some embodiments, the normalizing generates a set of chemical identifications for each mass spectrometry dataset in the plurality of mass spectrometry datasets. In some embodiments, the set of chemical identifications comprises a set of protein group identifications. In some embodiments, the normalizing comprises assigning a first peptide identification in a first mass spectrometry dataset in the plurality of mass spectrometry datasets and a second peptide identification in a second mass spectrometry dataset in the plurality of mass spectrometry datasets to the same protein group. The set of chemical identifications can be generated using a database comprising a plurality of chemicals and a corresponding plurality of mass spectrometry signals for the plurality of chemicals. The database can be generated based on genomic information obtained from an organism associated with one or more mass spectrometry datasets in the plurality of mass spectrometry datasets. In some embodiments, the database comprises polyamino acid sequences, functional information for polyamino acids, proteoforms for polyamino acids, mass spectra for polyamino acids, or any combination thereof. The database can provide one or more polyamino acids that creates a signal in a mass spectrometry dataset when detected by a mass spectrometer. By matching the signal to a polyamino acid, the mass spectrometry dataset can be used to generate a list of polyamino acids that are detected in a sample. In some cases, databases can provide functional annotations for a plurality of polyamino acids. For example, information about involvement of a polyamino acid in a biochemical pathway can be determined using a database. Appropriate databases can include UniProt, Wikipathways, Protein Data Bank, InterPro, The Human Protein Atlas, Kyoto Encyclopedia of Genes and Genomes, The Comprehensive Resource of Mammalian Protein Complexes (CORUM), Reactome Pathway Database, or any combination thereof. In some cases, protein groups can be determined using a protein grouping algorithm. A protein group can refer to one or more proteins that are identified by a set of shared peptide sequences. A protein group can comprise a master protein, wherein the master protein comprises the entire set of shared peptide sequences. A protein group may comprise additional proteins, wherein the additional proteins may be identified by the entire set or a subset of the shared peptide sequences. A set of shared peptide sequences can have one or more peptide sequences. Each peptide sequence can be in one or more sets of shared peptide sequences.

In a mass spectrometry experiment, a plurality of peptide sequences can be identified. The plurality of peptide sequences can be resolved such that the number of master proteins (thus the number of protein groups) is minimized given the information of the plurality of peptide sequences. The plurality of peptide sequences can be analyzed find the largest protein sequences possible from the given information.

In some cases, a peptide or a protein sequence may comprise amino acid sequences. In some cases, an amino acid sequence may comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some cases, a peptide or a protein sequence may further comprise post-translational modification. In some cases, a post-translation modification may comprise: acylation, myristoylation, palmitoylation, isoprenylation, farnesylation, geranylgeranylation, glypiation, phosphorylation, or any combination thereof. In some cases, a peptide or a protein sequence may further comprise a charge state of an amino acid in a sequence (e.g., aspartate/aspartic acid or glutamate/glutamic acid). In some cases, a peptide or a protein sequence may further comprise unnatural amino acids.

Various algorithms can be used to generate the set of peptide identifications and/or the set of protein group identifications. The algorithm can include an algorithm from ProteinProphet™, Protein Group Code Algorithm, MaxQuant, Comet, MSFragger, for some examples. The determining can comprise minimizing an objective function, using a computing node in the plurality of computing nodes, based on a pair of mass spectrometry datasets in the plurality of mass spectrometry datasets. In some embodiments, the determining comprises minimizing the objective function a unique pair of mass spectrometry datasets in the plurality of mass spectrometry datasets for each computing node in the plurality of computing nodes.

### Biological Sample

Mass spectrometry datasets can be generated by assaying one or more biological samples. In some embodiments, a biological sample may comprise a cell or be cell-free sample. In some embodiments, a biological sample may comprise a biofluid, such as blood, serum, plasma, urine, or cerebrospinal fluid (CSF). In some embodiments, a biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract or a cell-free, cell culture medium. In some embodiments, a biological sample may be obtained from a subject. In some embodiments, the subject may be a human or a non-human. In some embodiments, the subject may be a plant, a fungus, or an archaeon. In some embodiments, a biological sample can contain a plurality of proteins or proteomic data, which may be analyzed after adsorption or binding of proteins to the surfaces of the various sensor element (e.g., particle) types in a panel and subsequent digestion of protein coronas.

In some embodiments, the plurality of samples comprises at least 500, 5000, or 50000 samples. In some embodiments, the plurality of samples comprises at most 5000, 50000, 500000 samples. In some embodiments, the plurality of samples comprises a complex sample. In some embodiments, the complex sample comprises at least 100, 1000, 10000, 100000, or 1000000 unique biomolecules. In some embodiments, the complex sample comprises at least 100, 1000, 10000, 100000, or 1000000 unique proteins. In some embodiments, the complex sample comprises at most 1000, 10000, 100000, 1000000, or 10000000 unique biomolecules. In some embodiments, the complex sample comprises at most 1000, 10000, 100000, 1000000, or 10000000 unique proteins. In some embodiments, the complex sample comprises a biomolecule comprising at least about 0.1, 1, 10, 100, or 1000 kiloDaltons (kDa) in molecular weight. In some embodiments, the complex sample comprises a biomolecule comprising at most about 1, 10, 100, 1000, or 10000 kiloDaltons (kDa) in molecular weight. The systems and methods of the present disclosure can allow processing of larger datasets than currently available.

In some embodiments, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, a biological sample may comprise multiple biological samples (e.g., pooled plasma from multiple subjects, or multiple tissue samples from a single subject). In some embodiments, a biological sample may comprise a single type of biofluid or biomaterial from a single source.

In some embodiments, a biological sample may be diluted or pre-treated. In some embodiments, a biological sample may undergo depletion (e.g., the biological sample comprises serum) prior to or following contact with a surface disclosed herein. In some embodiments, a biological sample may undergo physical (e.g., homogenization or sonication) or chemical treatment prior to or following contact with a surface disclosed herein. In some embodiments, a biological sample may be diluted prior to or following contact with a surface disclosed herein. In some embodiments, a dilution medium may comprise buffer or salts, or be purified water (e.g., distilled water). In some embodiments, a biological sample may be provided in a plurality partitions, wherein each partition may undergo different degrees of dilution. In some embodiments, a biological sample may comprise may undergo at least about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

In some embodiments, the biological sample may comprise a plurality of biomolecules. In some embodiments, a plurality of biomolecules may comprise polyamino acids. In some embodiments, the polyamino acids comprise peptides, proteins, or a combination thereof. In some embodiments, the plurality of biomolecules may comprise nucleic acids, carbohydrates, polyamino acids, or any combination thereof. In some embodiments, a polyamino acid may be a proteolytic peptide. In some embodiments, a polyamino acid may be a tryptic peptide. In some embodiments, a polyamino acid may be a semi-tryptic peptide. A biological sample may comprise a member of any class of biomolecules, where “classes” may refer to any named category that defines a group of biomolecules having a common characteristic (e.g., proteins, nucleic acids, carbohydrates).

### Assays

In some embodiments, the computer-implemented method comprises performing a plurality of assays on the plurality of samples to generate the plurality of mass spectrometry datasets.

In some embodiments, the plurality of assays comprises selectively enriching a plurality of chemicals in the plurality of samples. In some embodiments, the selectively enriching comprises contacting the plurality of samples with a surface. In some embodiments, the selectively enriching comprises contacting the plurality of samples with a plurality of surfaces. In some embodiments, the selectively enriching comprises contacting the plurality of samples with a plurality of surfaces comprising distinct surface chemistries. In some embodiments, the contacting adsorbs the plurality of chemicals on the surface. In some embodiments, the contacting non-specifically binds the plurality of chemicals on the surface. In some embodiments, the surface comprises a particle surface of a particle. In some embodiments, the contacting forms a corona on the particle surface. In some embodiments, the particle comprises a paramagnetic core.

In some embodiments, the plurality of chemicals comprises a dynamic range of at least about 1, 2, 3, 4, 5, 6, 7, 8, 9, 10, 11, 12, 13, 14, 15, 16, 17, 18, or 19. In some embodiments, the plurality of chemicals comprises a dynamic range of at most about 1, 2, 3, 4, 5, 6, 7, 8, 9, 10, 11, 12, 13, 14, 15, 16, 17, 18, or 19. In some embodiments, the plurality of chemicals, when adsorbed, comprises a dynamic range that is decreased by at least about 1, 2, 3, 4, 5, 6, 7, 8, 9, 10, 11, 12, 13, 14, or 15 magnitudes. In some embodiments, the selectively enriching comprises releasing the plurality of chemicals from the surface. In some embodiments, the plurality of assays comprises performing mass spectrometry on the plurality of samples.

In some cases, the plurality of chemicals can be assayed using non-specific binding. A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle, or at most 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration.

### Proteomic Analysis

As used herein, “proteomic analysis”, “protein analysis”, and the like, may refer to any system or method for analyzing proteins in a sample, including the systems and methods disclosed herein. The present disclosure provides systems and methods for assaying using one or more surface. In some cases, a surface may comprise a surface of a high surface-area material, such as nanoparticles, particles, microparticles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a secretome or exosome). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated, or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate particle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may be comprise a protein, polynucleotide, or polysaccharide, or may be comparable in size to any of the aforementioned classes of species. In some cases, A surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle, or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

In some cases, the panels disclosed herein can be used to identify a number of proteins, peptides, protein groups, or protein classes using a protein analysis workflow described herein (e.g., a protein corona analysis workflow). In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of approaches, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, an assay may rapidly generate biological samples for analysis. In some cases, the biological samples may comprise proteolytic peptides. In some cases, beginning with an input biological sample (e.g., a buccal or nasal smear, plasma, secretome, or tissue), a method of the present disclosure may generate the biological samples in less than about 1, 2, 3, 4, 5, 6, 7, 8, 12, 16, 20, 24, or 48 hours. In some cases, beginning with an input biological sample (e.g., a buccal or nasal smear, plasma, secretome, or tissue), a method of the present disclosure may generate the biological samples in less than about 1, 2, 3, 4, 5, 6, 7, 8, 12, 16, 20, 24, or 48 hours.

In some cases, an assay may rapidly generate and analyze proteomic data. In some cases, beginning with an input biological sample (e.g., a buccal or nasal smear, plasma, or tissue), a method of the present disclosure may generate and obtain proteomic data in less than about 1, 2, 3, 4, 5, 6, 7, 8, 12, 16, 20, 24, or 48 hours. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. A particle of the present disclosure may be a nanoparticle. A nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. A nanoparticle may be less than 1000 nm in diameter. A particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. A microparticle may be less than 1000 μm in diameter. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

### Proteomic Information

In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof.

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and/or introns. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof. In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

### Machine Learning

In some embodiments, mass spectrometry datasets (including harmonized mass spectrometry datasets) can be processed using a machine learning algorithm. In some embodiments, identifications of biomolecules may be processed using a machine learning algorithm. In some embodiments, the identifications of biomolecules may comprise identifications of nucleic acids, variants thereof, proteins, variants thereof, and any combination thereof. In some embodiments, the machine learning algorithm may be an unsupervised or self-supervised learning algorithm. In some embodiments, the machine learning algorithm may be trained to learn a latent representation of the identifications of the biomolecules. In some embodiments, the machine learning algorithm may be supervised learning algorithm. In some embodiments, the machine learning algorithm may be trained to learn to associate a given set of identifications with a value associated with a predetermined task. For example, the predetermined task may comprise determining a disease state associated with the given set of identifications, where the value may indicate the probability of the disease state being present in a subject associated with the given set of identifications. A machine learning algorithm can be trained to identify a correlation between signals in a mass spectrometry dataset and a biological state. The trained machine learning algorithm can be used to identify a biomarker for the biological state.

In some embodiments, the method of determining a set of biomolecules associated with the disease or disorder and/or disease state can include the analysis of the biomolecule corona of at least two samples. This determination, analysis or statistical classification can be performed by methods, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, among others. In other words, the biomolecules in the corona of each sample are compared/analyzed with each other to determine with statistical significance what patterns are common between the individual corona to determine a set of biomolecules that is associated with the disease or disorder or disease state.

In some embodiments, machine learning algorithms can be used to construct models that accurately assign class labels to examples based on the input features that describe the example. In some case it may be advantageous to employ machine learning and/or deep learning approaches for the methods described herein. For example, machine learning can be used to associate the biomolecule corona with various disease states (e.g. no disease, precursor to a disease, having early or late stage of the disease, etc.). For example, In some embodiments, one or more machine learning algorithms can be employed in connection with the methods disclosed hereinto analyze data detected and obtained by the biomolecule corona and sets of biomolecules derived therefrom. For example, machine learning can be coupled with genomic and proteomic information obtained using the methods described herein to determine not only if a subject has a pre-stage of cancer, cancer or does not have or develop cancer, and also to distinguish the type of cancer.

In some embodiments, machine learning algorithms may also be used to associate the results from protein corona analysis and results from nucleic acid sequencing analysis and further associate any trends or correlations between proteins and nucleic acids to a biological state (e.g., disease state, health state, subtypes of disease such as stages of disease are cancer subtypes).

In some embodiments, machine learning may be used to cluster proteins detected using a plurality of surfaces. In some embodiments, a panel of surfaces may be used to assay proteins from one or more biological samples. In some embodiments, a surface in the panel of surfaces may comprise diverse physicochemical properties. In some embodiments, proteins detected by the panel of surfaces may be clustered using a clustering algorithm. In some embodiments, proteins detected by the panel of surfaces may be clustered based at least partially on the intensities of detected protein signals, particle chemical properties, protein structural and/or functional groups, or any combination thereof.

A panel of surfaces may comprise any number of surfaces. In some embodiments, a panel of surfaces may comprise at least about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces. In some embodiments, a panel of surfaces may comprise at most about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces. In some embodiments, the panel has 2 to 10 surfaces, 2 to 5 surfaces, or 3 to 7 surfaces.

Inputs to a machine learning algorithm may comprise various kinds of inputs. In some embodiments, an input may comprise a value that represents a physicochemical property of a surface used to assay a biomolecule. A physicochemical property of a particle may comprise various properties disclosed herein, which includes: charge, hydrophobicity, hydrophilicity, amphipathicity, coordinating, reaction class, surface free energy, various functional groups/modifications (e.g., sugar, polymer, amine, amide, epoxy, crosslinker, hydroxyl, aromatic, or phosphate groups). In some embodiments, an input may comprise a value that represents a parameter of a given assay. A parameter may comprise incubation conditions including temperature, incubation time, pH, buffer type, and any variables in performing an assay disclosed herein.

In some embodiments, a clustering algorithm can refer to a method of grouping samples in a dataset by some measure of similarity. In some embodiments, samples can be grouped in a set space, for example, element ‘a’ is in set ‘A’. In some embodiments, samples can be grouped in a continuous space, for example, element ‘a’ is a point in Euclidean space with distance ‘l’ away from the centroid of elements comprising cluster ‘A’. In some embodiments, samples can be grouped in a graph space, for example, element ‘a’ is highly connected to elements comprising cluster ‘A’. In some embodiments, clustering can refer to the principle of organizing a plurality of elements into groups in some mathematical space based on some measure of similarity.

In some embodiments, clustering can comprise grouping any number of biomolecules in a dataset by any quantitative measure of similarity. In some embodiments, clustering can comprise K-means clustering. In some embodiments, clustering can comprise hierarchical clustering. In some embodiments, clustering can comprise using random forest models. In some embodiments, clustering can comprise boosted tree models. In some embodiments, clustering can comprise using support vector machines. In some embodiments, clustering can comprise calculating one or more N−1 dimensional surfaces in N-dimensional space that partitions a dataset into clusters. In some embodiments, clustering can comprise distribution-based clustering. In some embodiments, clustering can comprise fitting a plurality of prior distributions over the data distributed in N-dimensional space. In some embodiments, clustering can comprise using density-based clustering. In some embodiments, clustering can comprise using fuzzy clustering. In some embodiments, clustering can comprise computing probability values of a data point belonging to a cluster. In some embodiments, clustering can comprise using constraints. In some embodiments, clustering can comprise using supervised learning. In some embodiments, clustering can comprise using unsupervised learning.

In some embodiments, clustering can comprise grouping biomolecules based on similarity. In some embodiments, clustering can comprise grouping biomolecules based on quantitative similarity. In some embodiments, clustering can comprise grouping biomolecules based on one or more features of each protein. In some embodiments, clustering can comprise grouping biomolecules based on one or more labels of each protein. In some embodiments, clustering can comprise grouping biomolecules based on Euclidean coordinates in a numerical representation of biomolecules. In some embodiments, clustering can comprise grouping biomolecules based on protein structural groups or functional groups (e.g., protein structures, substructures, or functional groups from protein databases such as Protein Data Bank or CATH Protein Structure Classification database). In some embodiments, a protein structural group or functional group may comprise protein primary structure, secondary structure, tertiary structure, or quaternary structure. In some embodiments, a protein structural group or functional group may be based at least partially on alpha helices, beta sheets, relative distribution of amino acids with different properties (e.g., aliphatic, aromatic, hydrophilic, acidic, basic, etc.), a structural families (e.g., TIM barrel and beta barrel fold), protein domains (e.g., Death effector domain). In some embodiments, a protein structural group or functional group may be based at least partially on functional or spatial properties (e.g., functional groups-group of immune globulins, cytokines, cytoskeletal biomolecules, etc.).

### Computer Systems

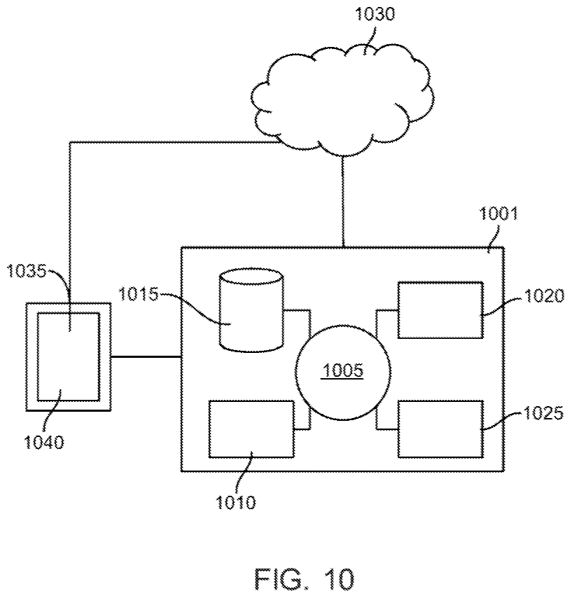

The present disclosure provides computer systems that are programmed to implement methods of the disclosure. FIG. 10 shows a computer system 1001 that is programmed or otherwise configured to, for example, analyze, convert, and/or display omics data.

The computer system 1001 may regulate various aspects of analysis, calculation, and generation of the present disclosure, such as, for example, converting, analyzing, and/or displaying omics data. The computer system 1001 may be an electronic device of a user or a computer system that is remotely located with respect to the electronic device. The electronic device may be a mobile electronic device.

The computer system 1001 includes a central processing unit (CPU, also “processor” and “computer processor” herein) 1005, which may be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 may be a data storage unit (or data repository) for storing data. The computer system 1001 may be operatively coupled to a computer network (“network”) 1030 with the aid of the communication interface 1020. The network 1030 may be the Internet, an internet and/or extranet, or an intranet and/or extranet that is in communication with the Internet.

The network 1030 in some cases is a telecommunication and/or data network. The network 1030 may include one or more computer servers, which may enable distributed computing, such as cloud computing. For example, one or more computer servers may enable cloud computing over the network 1030 (“the cloud”) to perform various aspects of analysis, calculation, and generation of the present disclosure, such as, for example, converting, analyzing, and/or displaying omics data. Such cloud computing may be provided by cloud computing platforms such as, for example, Amazon Web Services (AWS), Microsoft Azure, Google Cloud Platform, and IBM cloud. The network 1030, in some cases with the aid of the computer system 1001, may implement a peer-to-peer network, which may enable devices coupled to the computer system 1001 to behave as a client or a server.

The CPU 1005 may comprise one or more computer processors and/or one or more graphics processing units (GPUs). The CPU 1005 may execute a sequence of machine-readable instructions, which may be embodied in a program or software. The instructions may be stored in a memory location, such as the memory 1010. The instructions may be directed to the CPU 1005, which may subsequently program or otherwise configure the CPU 1005 to implement methods of the present disclosure. Examples of operations performed by the CPU 1005 may include fetch, decode, execute, and writeback.

The CPU 1005 may be part of a circuit, such as an integrated circuit. One or more other components of the system 1001 may be included in the circuit. In some embodiments, the circuit is an application specific integrated circuit (ASIC).

The storage unit 1015 may store files, such as drivers, libraries and saved programs. The storage unit 1015 may store user data, e.g., user preferences and user programs. The computer system 1001 in some cases may include one or more additional data storage units that are external to the computer system 1001, such as located on a remote server that is in communication with the computer system 1001 through an intranet or the Internet.

The computer system 1001 may communicate with one or more remote computer systems through the network 1030. For instance, the computer system 1001 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 1001 via the network 1030.

Methods as described herein may be implemented by way of machine (e.g., computer processor) executable code stored on an electronic storage location of the computer system 1001, such as, for example, on the memory 1010 or electronic storage unit 1015. The machine executable or machine readable code may be provided in the form of software. During use, the code may be executed by the processor 1005. In some embodiments, the code may be retrieved from the storage unit 1015 and stored on the memory 1010 for ready access by the processor 1005. In some situations, the electronic storage unit 1015 may be precluded, and machine-executable instructions are stored on memory 1010.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 1001, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 may include or be in communication with an electronic display 1035 that comprises a user interface (UI) 1040 for providing, for example, converting, analyzing, and/or displaying omics data. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 1005. The algorithm can, for example, converting, analyzing, and/or displaying omics data.

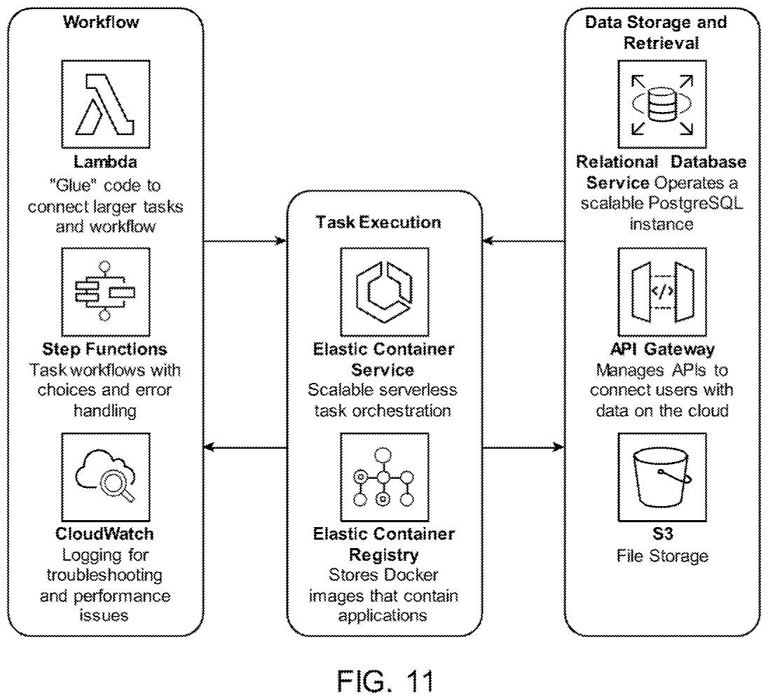

FIG. 11 schematically illustrates a cloud-based distributed computing environment, in accordance with some embodiments. In some embodiments, a computer system or a computer-implemented method of the present disclosure are configured to perform instructions on an event-driven and serverless platform. In some embodiments, instructions are performed with concurrency. In some embodiments, instructions are performed with scaling controls. In some embodiments, instructions can be packaged in container images. The container images can be configured to run on a variety of computing environments. In some embodiments, instructions comprise a signature for verifying integrity of the instructions. In some embodiments, instructions comprise a database proxy. The database proxy can manage a plurality of database connections and relay a query from an instruction to a database. In some embodiments, instructions can store or retrieve datasets from an elastic storage system, a local storage system, or both. In some embodiments, instructions comprise one or more states that indicate which instruction was last performed and/or which instruction is to be performed next. In some embodiments, instructions automatically logs events (e.g., errors or performance issues) that occur while the instructions are performed.

Containers for instructions can be deployed on serverless computing instance. A first subset of the instructions can be retrieved and used on a first instance. A second subset of the instructions can be retrieved and used on a second instance. The first subset of the instructions and the second subset of the instructions can be orchestrated to be performed together using the first instance and the second instance in parallel. The size of the first instance and the second instance can be based on the complexity of the first subset of instructions, the second subset of instructions, the amount of the dataset to be processed, or any combination thereof.

Datasets can be stored and retrieved from a variety of storage systems. In some embodiments, a storage system can be a relational database. In some embodiments, a storage system can be a non-relational database. In some embodiments, a storage system can be a distributed database. In some embodiments, a storage system can be an object-based database.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Cloud Scalable Omics Data Analysis Pipeline

This example describes provides a platform of the present disclosure. The platform is configured to automatically transfer LC-MS/MS datasets to the cloud, and convert the LC-MS/MS dataset to standard mzML, parquet, and HDF5 filetypes. Single files for every LC-MS injection result is analyzed automatically upon raw data file arrival. Group run analyses can be user-specified with pre-defined recipes and settings (e.g., using the Fragpipe workflow in the AWS environment). The analysis can be performed on at least 1000 files. The analysis can be performed on at least 1000 samples. The platform can employ Spark-accelerated modular workflows built on top of open-source Alphapept.

The platform can use a serverless task infrastructure (e.g., introducing a cloud scalable pipeline using AWS Step functions). A high level of scalability can be achieved by containerizing legacy applications and orchestrating them in cloud environments (e.g., using AWS ECS and Step Functions). Automated cloud-connected data analysis can be used to analyze outputs from a fleet of MS instruments from multiple vendors, generating terabyte-scale data annually.

A cloud scalable omics data analysis pipeline can begin with a Watchdog monitors that can transfer MS files, as they arrive, from one or more LC-MS/MS instruments into AWS S3 file storage. The transfer can trigger Lambda Functions, which acts as a connection to one or more Step Functions, which maps out tasks, choices, and error-handling that may be necessary for the analysis of MS data. Elastic Container Service Tasks, which execute computationally rigorous code, can use Docker-containerized executables that can be instantiated using a mixture of AWS's Fargate and Batch. In some cases, Batch can be leveraged when Fargate's compute and local storage is not sufficient. Batch with Spot Instances may be leveraged for short but intense jobs to reduce costs. The cloud scalable omics data analysis pipeline outputs may be stored in a combination of S3 buckets, a non-relational Mongo database, and a relational PostgreSQL database, which can operate on a principle of polyglot persistence. In some cases, differently structured data may be stored in different types of databases. In some cases, highly structured experimental data may be stored in a relational PostgreSQL database (SeerDB). Instrument readings and quality control data can be stored in non-relational MongoDB database. APIs and various internal applications can be used to query one or more datastores to return information collectively. In some embodiments, the cloud scalable omics data analysis pipeline may comprise massively parallel group run contexts.

Seer's current database contains at least about 500 terabytes of raw, semi-structured and structured data from a fleet of LC-MS/MS instruments from multiple vendors. Peptide and protein annotations are query-able using a polyglot persistence model of document and relational systems. Thousands of peptide and protein annotations are query-able using a polyglot persistence model of document and relational systems. Cloud-first laboratory pipes data using an Amazon Web Services (AWS) storage gateway can service and automatically process raw data using event based-triggering mechanisms. Users may also launch group analysis runs with pre-defined recipes. The described architecture may rely on open source algorithm components. In some embodiments, the cloud scalable omics data analysis pipeline may analyze thousands of samples in hours. The cloud scalable omics data analysis pipeline may support hundreds of terabytes of incoming LCMS data, annually. The cloud scalable omics data analysis pipeline may process at least about 150 files with 140 AWS Batch jobs per day. The cloud scalable omics data analysis pipeline can process at least about 2600 AWS Fargate tasks per day.

### Example 2: Large Scale, Cloud Enabled Re-Analysis

In an example, the Proteograph™ technology may be applied to cancer cohorts (e.g., including cohorts of more than 200 samples, or more than 1000 samples) to identiy protein groups across an entire cohort. Data was acquired in data-independent-acquisition (DIA) mode on a Sciex Triple TOF 6600+ with EKSPERT nano-LC 425 LC running a 33 min gradient. Previously, computational resources limited large-scale group analysis of the data, but using new scalable cloud infrastructure enabled processing of the entire cohort in one large group-run using DIA-NN v1.8 in library free mode using the—relaxed-prot-inf flag. Downstream analysis, including variational autoencoder (VAE) neural network, may be built on top of open-source python libraries.

Large-scale re-analysis yielded nearly 4,000 protein groups across the entire cohort with each sample averaging over 2,000 protein groups. This corresponds to about a 5-fold increase in depth compared to neat plasma, which may be around 400 protein groups per sample. This corresponded to nearly 25% increase per sample from a prior analysis. The increased depth may be due to a combination of more sensitive library-free search and a large group run combining all the injections. Injections may be combined after acquisition (e.g., MS acquired spectrums). Cloud-based architecture may enable protein grouping through combining multiple injections to create the most comparable group.

### Example 3: Cloud-Scalable Method for Protein Inference

False Discovery Rate (FDR) controlled protein identification results can use several processes. Some processes can be highly parallel and scales easily with larger and larger datasets. For example, in feature finding, peptide spectrum matches (PSMs) and uniquely identified peptides are generated from each individual injection. Feature finding can be rather flexible as they may be run as an individual file, multiple files on the same machine, or different files on different machines in parallel (e.g. Fargate). As another example, searching (e.g., using the MSFragger search engine component of Fragpipe) may process two thousand files in a few hours using autoscaling features of AWS batch or Fargate.

Bottlenecks may appear in some processes where data aggregation is performed. For example, protein inference (e.g., using Protein Prophet™) adds significant overhead (e.g., days) to the processing time to process even on a large vertically-scaled instance.

During protein inferences, results from all runs are pooled and analyzed simultaneously, which can strain both memory and compute. For example, in an MsFragger group run of over 2300 injections, this process, using Protein Prophet™, takes over 30 hours, which is far more than half of the total runtime in this example.

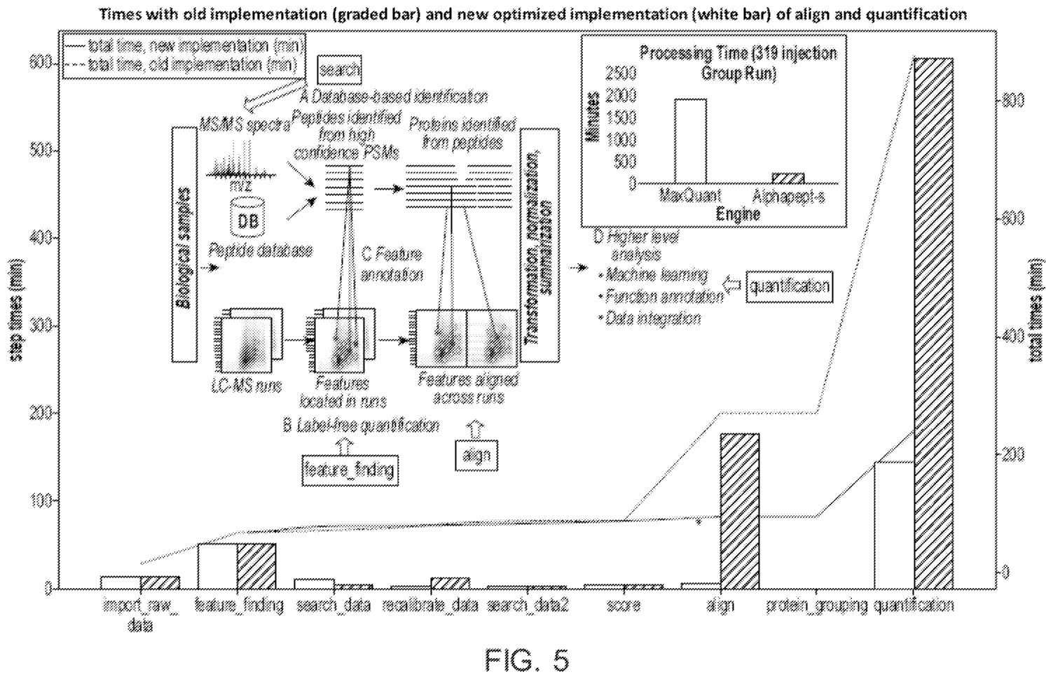

This example describes performing single cohort Label-Free quant analysis (group run) of more than 400 samples, which includes 2000+LC-MS/MS injections, to produce over 5300 protein groups in under 48 hours of compute time when components of the workflow (e.g., using the Fragpipe) are deployed in a cloud computing environment. Bottleneck analysis of the pipeline revealed protein inference being a major contributor, as shown in FIG. 5.

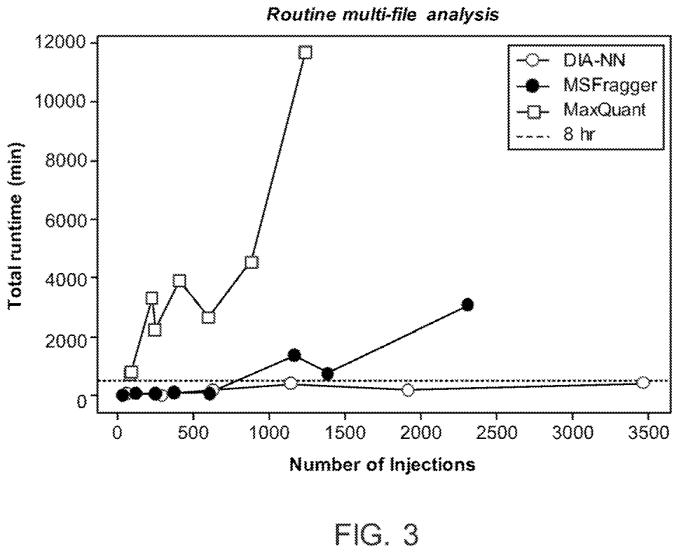

FIG. 3 shows a plot of total runtime as a function of the number of injections analyzed, in accordance with some embodiments. DIA-NN can process thousands of samples in under 8 hours (without tMBR). However, scaling beyond about 5000 samples approaches a computational cost that can benefit significantly from harmonization of datasets and modularization of analysis pipelines.

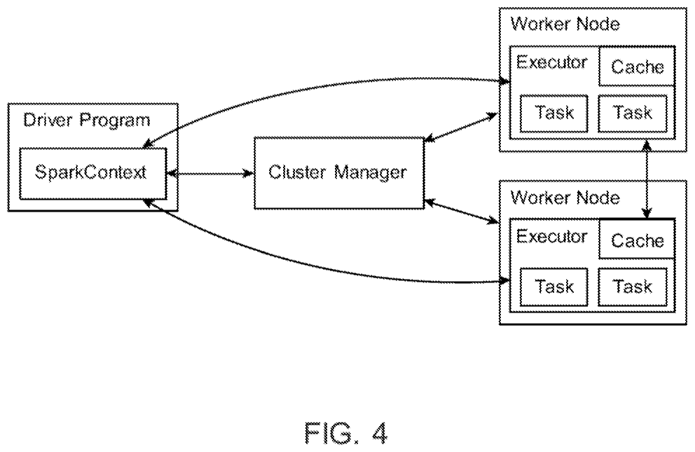

Provided is an alternative protein inference algorithm deployed on an integrated distributed compute framework to mitigate significant bottlenecks in protein inference. FIG. 4 schematically illustrates a method for distributing cached dataset and tasks, in accordance with some embodiments. Using Apache Spark, the deployment exceeds vertical scalability limited by legacy implementations. The deployment is integrated with a cloud computing infrastructure through API design. These components may be made to seamlessly interact, and more complex and scalable pipelines may be created.

A protein/peptide graph network and a razor approach (Tyanova et al, 2016) is used for protein inference in this deployment, which is used in MaxQuant, Alphapept, and other engines. The approach aims to solve a protein inference problem by creating a network with connections between all peptides and proteins, the proteins with the most peptide connections may be iteratively selected as the “razor protein” and removed from the graph. This greedy approach may be a simpler solution than PeptideProphet™'s approach, enables a design for a distributed approach that reduces the computational bottleneck.

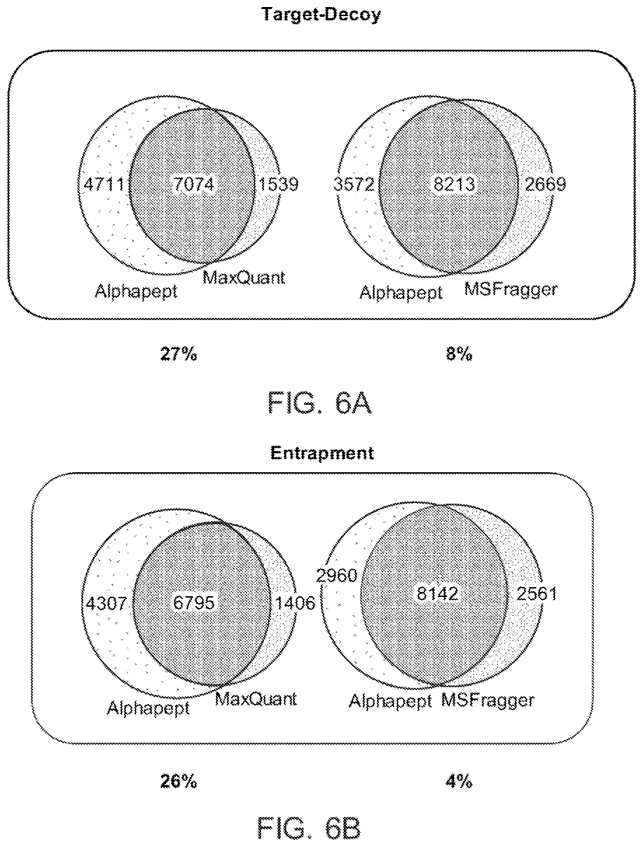

FIG. 5 shows the computational costs for different processes in a label-free quantification analysis pipeline, in accordance with some embodiments. Most significant overhead was observed in the alignment and the quantification. By using the distributed approach disclosed herein, significant savings in time was observed for the alignment and the quantification. Overall, about 10 hours were saved in the new implementation from the 15 hours of the old implementation. FIGS. 6A-6B show the number of peptides identified using target-decoy and entrapment analysis, in accordance with some embodiments. Using target-decoy and entrapment analysis, improvements are shown in both speed and sensitivity compared with other search engine.

TABLE 1
Target-decoy and entrapment analysis results.

| | Unique Peptides | False Match Rate (FMR) |
|---|---|---|
| MaxQuant | 8201 | 0 |
| MSFragger | 10703 | 0.005 |
| Alphapept | 11102 | 0.02 |

### Example 4: Harmonizing and Broadcasting Mass Spectrometry Data Across Computating Nodes

In some cases, mass spectrometry data is represented in a file comprising a binary memory mapped database that contains the raw signal information from an LC-MS/MS run. The raw signal information comprises an array of LC-MS/MS scans across time. Each file can be viewed as a ‘data cubes’ that comprises arrays of scan information, the elements of which are discretized samples of a chromatographic run. Each ‘scan’ comprises an array of mass-to-charge peaks and their corresponding retention time and intensity. The files are often in one of various proprietary binary format from various vendors, and are configured to be read only using vendor software or utilities that the vendors provide.

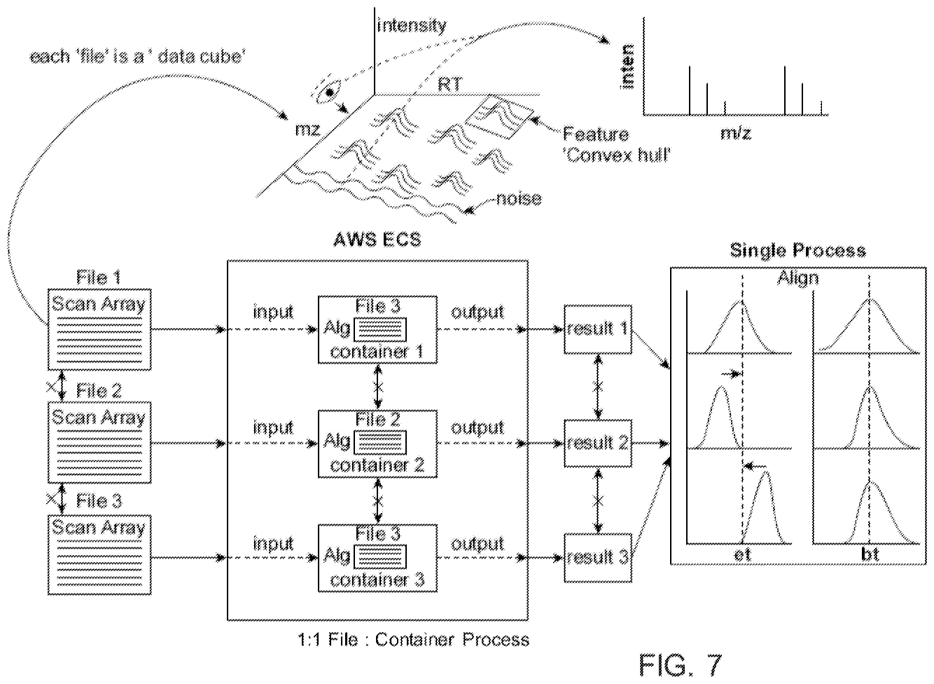

FIG. 7 schematically illustrates a process for performing alignment based on mass spectrometry datasets, in accordance with some embodiments. Since mass spectrometry data arising from each injection can be independent of data from another injection, each ‘file’ can also be independent of another. As such, any processing that needs to be done must happen independently of another. In a cloud computing environment (e.g., AWS environment) one can ‘clone a process’, where an algorithm runs on an individual file and then auto scales the containers for the algorithms based on the number of input files. In this example, there are three input files and thus three separate containers running a data filtering algorithm for each file in parallel. The three input files may be processed independently, and the parallelization is so called “embarrassingly” parallel that is the maximum extent of the parallelization. Note that each file will also have a single output.

However, if a data aggregation is needed, then all of these outputs must reside in a single location to be processed together (e.g., in the case of chromatographic alignment). In chromatographic alignment, features from separate output files are compared with each other and aligned. This can be a computational bottleneck for scaling the size of the data, since as the number of input files is increased, the size of the data that is needed to reside on a single processing node can be limited to a single machine's hardware and memory.

In some embodiments, two elements are applied to scale beyond single machine hardware limitation where data aggregation is performed (e.g., Alignment and match-between-runs (MBR)).

1. HDF5/Parquet file formatting

2. Spark and broadcasting

The binary files (which contain the scan array data) are converted to the HDF5 format. Vendor-provided utilities are used to extract the scan data in the binary files and then they are stored as HDF5 containers. Once all the data is now in the HDF5 format, all the data at once (i.e., all files in a single bucket) via the Spark interface. The data comprising all of the scans in all of the files can be viewed as a single large collection of scans. The “atomic unit” of the signals (scans) from LC-MS are comprised in an instance of one large collection. In a distributed computing system, blocks of scan data can be sent to different nodes in the cluster (as handled by Spark). Any processing function can be used to transform the data block and then aggregate the final results.

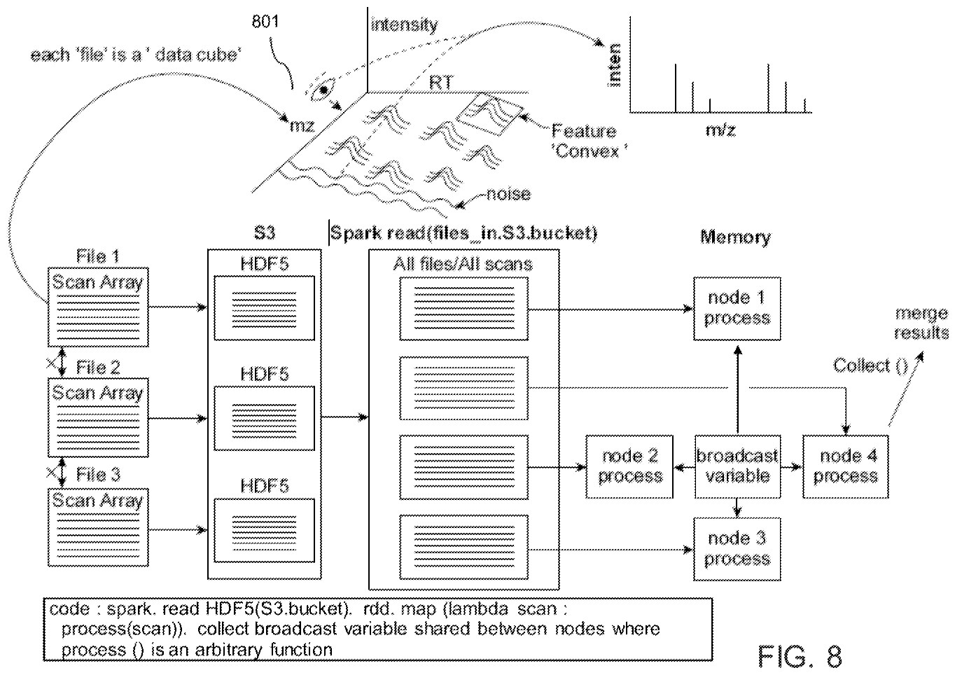

An example of broadcasting is demonstrated in FIG. 8, which illustrates a process for performing alignment based on harmonized mass spectrometry datasets, in accordance with some embodiments. Each file (which is now a collection of entities, in this case a collection of chromatographic features is now read once and stored as a broadcast variable. The mapping function is now each ‘file’ comparing their list of features against the broadcast variable (the abstraction of all files).

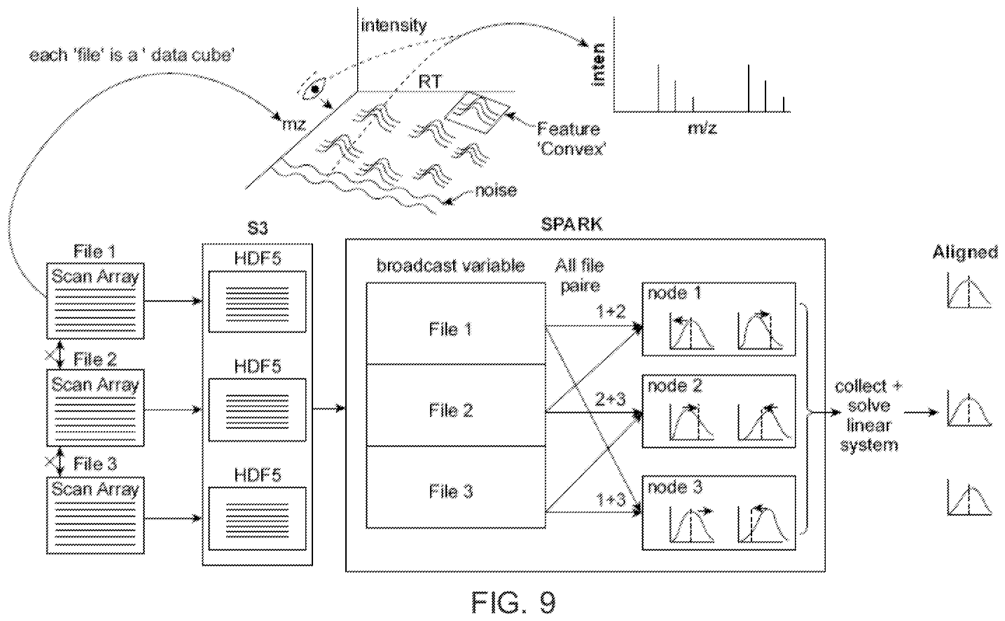

Another example of broadcasting is demonstrated in FIG. 9, which schematically illustrates a process for broadcasting mass spectrometry datasets that are converted to HDF5 format between computing nodes, in accordance with some embodiments.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A computer-implemented method for normalizing and processing mass spectrometry datasets, comprising: (a) obtaining a plurality of mass spectrometry datasets obtained from a plurality of samples; (b) loading the plurality of mass spectrometry datasets into a memory of a computing node to generate a cached dataset; (c) transmitting a copy of the cached dataset to a plurality of cache memories of a plurality of computing nodes; (d) determining, using the plurality of computing nodes, a plurality of feature values for the plurality of mass spectrometry datasets; (e) normalizing, using the plurality of computing nodes, across the plurality of mass spectrometry datasets using the plurality of feature values to generate a plurality of normalized mass spectrometry datasets; and (f) processing the plurality of normalized mass spectrometry datasets to compare the plurality of samples.

Embodiment 2. The computer-implemented method of Embodiment 1, wherein the plurality of mass spectrometry datasets comprises a set of precursors for each sample in the plurality of samples.

Embodiment 3. The computer-implemented method of Embodiment 2, wherein the set of precursors comprises a set of biomolecule precursors.

Embodiment 4. The computer-implemented method of Embodiment 3, wherein the set of biomolecule precursors comprises a set of polyamino acid precursors.

Embodiment 5. The computer-implemented method of any one of Embodiments 1-4, wherein the plurality of mass spectrometry datasets comprises a set of chemical identifications for each sample in the plurality of samples.

Embodiment 6. The computer-implemented method of Embodiment 5, wherein the set of chemical identifications comprises a set of biomolecule identifications.

Embodiment 7. The computer-implemented method of Embodiment 6, wherein the set of biomolecule identifications comprises a set of polyamino acid identifications.

Embodiment 8. The computer-implemented method of Embodiment 7, wherein the set of polyamino acid identifications comprises a set of tryptic or semi-tryptic peptide identifications.

Embodiment 9. The computer-implemented method of any one of Embodiments 5-8, wherein the plurality of mass spectrometry datasets comprises a set of chemical intensities for each sample in the plurality of samples.

Embodiment 10. The computer-implemented method of Embodiment 9, wherein the set of chemical intensities comprises a set of biomolecule intensities.

Embodiment 11. The computer-implemented method of Embodiment 10, wherein the set of biomolecule intensities comprises a set of polyamino acid intensities.

Embodiment 12. The computer-implemented method of Embodiment 11, wherein the set of polyamino acid intensities comprises a set of tryptic or semi-tryptic peptide intensities.

Embodiment 13. The computer-implemented method of any one of Embodiments 7-12, wherein the set of polyamino acid identifications comprises a set of protein group identifications.

Embodiment 14. The computer-implemented method of Embodiment 13, wherein the set of polyamino acid intensities comprises a set of protein group intensities.

Embodiment 15. The computer-implemented method of any one of Embodiments 1-14, wherein the plurality of mass spectrometry datasets comprises a data independent acquisition (DIA) mass spectrometry dataset, a data dependent acquisition (DDA) mass spectrometry dataset, or both.

Embodiment 16. The computer-implemented method of any one of Embodiments 1-15, wherein the plurality of mass spectrometry datasets comprises a LC-MS dataset, a LC-MS/MS dataset, or both.

Embodiment 17. The computer-implemented method of any one of Embodiments 1-16, wherein the plurality of samples comprises at least 500, 5000, or 50000 samples.

Embodiment 18. The computer-implemented method of any one of Embodiments 1-17, wherein the plurality of samples comprises at most 5000, 50000, 500000 samples.

Embodiment 19. The computer-implemented method of any one of Embodiments 1-18, wherein the plurality of samples comprises a complex sample.

Embodiment 20. The computer-implemented method of Embodiment 19, wherein the complex sample comprises a biological sample.

Embodiment 21. The computer-implemented method of Embodiment 20, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 22. The computer-implemented method of Embodiment 21, wherein the biological sample comprises plasma or serum.

Embodiment 23. The computer-implemented method of any one of Embodiments 19-22, wherein the complex sample comprises at least 100, 1000, 10000, 100000, or 1000000 unique biomolecules.

Embodiment 24. The computer-implemented method of Embodiment 23, wherein the complex sample comprises at least 100, 1000, 10000, 100000, or 1000000 unique proteins.

Embodiment 25. The computer-implemented method of any one of Embodiments 19-24, wherein the complex sample comprises at most 1000, 10000, 100000, 1000000, or 10000000 unique biomolecules.

Embodiment 26. The computer-implemented method of Embodiment 25, wherein the complex sample comprises at most 1000, 10000, 100000, 1000000, or 10000000 unique proteins.

Embodiment 27. The computer-implemented method of claim any one of Embodiments 19-26, wherein the complex sample comprises a biomolecule comprising at least about 0.1, 1, 10, 100, or 1000 kiloDaltons (kDa) in molecular weight.

Embodiment 28. The computer-implemented method of claim any one of Embodiments 19-27, wherein the complex sample comprises a biomolecule comprising at most about 1, 10, 100, 1000, or 10000 kiloDaltons (kDa) in molecular weight.

Embodiment 29. The computer-implemented method of any one of Embodiments 1-28, wherein the feature values are based on isotopic clusters.

Embodiment 30. The computer-implemented method of any one of Embodiments 1-29, wherein the feature values comprise retention time, mass-to-charge ratio, aggregate peak area of the isotope cluster, ion mobility, or any combination thereof.

Embodiment 31. The computer-implemented method of any one of Embodiments 1-30, wherein the normalizing generates a set of aligned precursors for each mass spectrometry dataset in the plurality of mass spectrometry datasets.

Embodiment 32. The computer-implemented method of Embodiment 31, further comprising identifying a first chemical from a first mass spectrometry dataset in the plurality of mass spectrometry datasets based on an aligned precursor in the set of aligned precursors of a second mass spectrometry dataset.

Embodiment 33. The computer-implemented method of Embodiment 31 or 32, wherein the plurality of feature values comprises a feature value for the set of precursors of each mass spectrometry dataset in the plurality of mass spectrometry datasets.

Embodiment 34. The computer-implemented method of Embodiment 33, wherein the feature value is configured for normalizing retention time, mass-to-charge ratio, ion mobility, or a combination thereof.

Embodiment 35. The computer-implemented method of Embodiment 34, wherein the feature value is a shifting value.

Embodiment 36. The computer-implemented method of any one of Embodiments 29-35, wherein the determining comprises minimizing an objective function, using a computing node in the plurality of computing nodes, based on a pair of mass spectrometry datasets in the plurality of mass spectrometry datasets.

Embodiment 37. The computer-implemented method of Embodiment 36, wherein the determining comprises minimizing the objective function for a unique pair of mass spectrometry datasets in the plurality of mass spectrometry datasets for each computing node in the plurality of computing nodes.

Embodiment 38. The computer-implemented method of any one of Embodiments 1-28, wherein the normalizing generates a set of relative abundances for each mass spectrometry dataset in the plurality of mass spectrometry datasets.

Embodiment 39. The computer-implemented method of Embodiment 38, wherein normalizing comprises label-free quantification.

Embodiment 40. The computer-implemented method of Embodiment 38 or 39, wherein the set of relative abundances comprises a set of chemical relative abundances.

Embodiment 41. The computer-implemented method of Embodiment 40, wherein the set of chemical relative abundances comprises a set of biomolecule relative abundances.

Embodiment 42. The computer-implemented method of Embodiment 41, wherein the set of biomolecule relative abundances comprises a set of polyamino acid relative abundances.

Embodiment 43. The computer-implemented method of Embodiment 41 or 42, wherein the set of chemical relative abundances represent relative abundances of chemicals between the plurality of mass spectrometry datasets.

Embodiment 44. The computer-implemented method of Embodiment 43, wherein the set of relative abundances represent relative abundances of polyamino acids between the plurality of mass spectrometry datasets.

Embodiment 45. The computer-implemented method of any one of Embodiments 38-44, wherein the plurality of feature values comprises a feature value for the set of chemical intensities of each mass spectrometry dataset in the plurality of mass spectrometry datasets.

Embodiment 46. The computer-implemented method of Embodiment 45, wherein the normalizing comprises adjusting the set of chemical intensities for each mass spectrometry dataset in the plurality of mass spectrometry datasets based on the plurality of feature values.

Embodiment 47. The computer-implemented method of any one of Embodiments 38-46, wherein the determining comprises minimizing an objective function, using a computing node in the plurality of computing nodes, based on a pair of mass spectrometry datasets in the plurality of mass spectrometry datasets.

Embodiment 48. The computer-implemented method of Embodiment 47, wherein the determining comprises minimizing the objective function for a unique pair of mass spectrometry datasets in the plurality of mass spectrometry datasets for each computing node in the plurality of computing nodes.

Embodiment 49. The computer-implemented method of Embodiment 48, wherein the objective function comprises:

$$L = {\sum }_{p}^{N} \vert \frac{I ( {Norm}_{A} , p )}{I ( {Norm}_{B} , p )} \vert ,$$

wherein N is a number of chemical identifications in the set of chemical identifications, wherein p is a chemical in the set of chemical identifications, wherein I is an intensity value for the set of chemical intensities, wherein Norm_{A} is a first feature value for a first mass spectrometry dataset in the pair of mass spectrometry datasets, and wherein Norm_{B} is a second feature value for a second mass spectrometry dataset in the pair of mass spectrometry datasets.

Embodiment 50. The computer-implemented method of Embodiment 49, wherein the objective function comprises:

$$L = {\sum }_{A , B}^{M} {\sum }_{p}^{N} \vert \frac{I ( {Norm}_{A} , p , A )}{I ( {Norm}_{B} , p , B )} \vert ,$$

wherein M is a number of unique pairs of mass spectrometry datasets in the plurality of mass spectrometry datasets, and wherein A,B is the unique pair of mass spectrometry datasets in the plurality of mass spectrometry datasets.

Embodiment 51. The computer-implemented method of any one of Embodiments 1-28, wherein the normalizing generates a set of chemical identifications for each mass spectrometry dataset in the plurality of mass spectrometry datasets.

Embodiment 52. The computer-implemented method of Embodiment 51, wherein the set of chemical identifications comprises a set of protein group identifications.

Embodiment 53. The computer-implemented method of Embodiment 52, wherein the normalizing comprises assigning a first peptide identification in a first mass spectrometry dataset in the plurality of mass spectrometry datasets and a second peptide identification in a second mass spectrometry dataset in the plurality of mass spectrometry datasets to the same protein group.

Embodiment 54. The computer-implemented method of Embodiment 53, wherein the determining comprises minimizing an objective function, using a computing node in the plurality of computing nodes, based on a pair of mass spectrometry datasets in the plurality of mass spectrometry datasets.

Embodiment 55. The computer-implemented method of Embodiment 54, wherein the determining comprises minimizing the objective function a unique pair of mass spectrometry datasets in the plurality of mass spectrometry datasets for each computing node in the plurality of computing nodes.

Embodiment 56. The computer-implemented method of any one of Embodiments 1-55, wherein a processing time for performing (b)-(f) is substantially linear as a function of a number of mass spectrometry datasets in the plurality of mass spectrometry datasets.

Embodiment 57. The computer-implemented method of any one of Embodiments 1-56, wherein performing (b)-(f) takes less than ax^{1.8} amount of compute time, wherein x is a number of mass spectrometry datasets in the plurality of mass spectrometry datasets, and wherein a is a constant.

Embodiment 58. The computer-implemented method of Embodiment 57, wherein performing (b)-(f) takes less than ax^{1.6} amount of compute time, wherein x is a number of mass spectrometry datasets in the plurality of mass spectrometry datasets, and wherein a is a constant.

Embodiment 59. The computer-implemented method of Embodiment 58, wherein performing (b)-(f) takes less than ax^{1.4} amount of compute time, wherein x is a number of mass spectrometry datasets in the plurality of mass spectrometry datasets, and wherein a is a constant.

Embodiment 60. The computer-implemented method of Embodiment 59, wherein performing (b)-(f) takes less than ax^{1.2} amount of compute time, wherein x is a number of mass spectrometry datasets in the plurality of mass spectrometry datasets, and wherein a is a constant.

Embodiment 61. The computer-implemented method of Embodiment 60, wherein performing (b)-(f) takes less than ax amount of compute time, wherein x is a number of mass spectrometry datasets in the plurality of mass spectrometry datasets, and wherein a is a constant.

Embodiment 62. The computer-implement method of any one of Embodiments 1-61, wherein the processing further comprises determining a biomarker based on the plurality of normalized mass spectrometry datasets.

Embodiment 63. The computer-implement method of any one of Embodiments 1-62, wherein the processing further comprises performing a power curve analysis based on the plurality of normalized mass spectrometry datasets.

Embodiment 64. The computer-implement method of any one of Embodiments 1-63, wherein the processing further comprises training a machine learning model based on the plurality of normalized mass spectrometry datasets.

Embodiment 65. The computer-implement method of any one of Embodiments 1-64, wherein the processing further comprises performing clustering analysis based on the plurality of normalized mass spectrometry datasets.

Embodiment 66. The computer-implemented method of any one of Embodiments 1-65, further comprising, before (a), performing a plurality of assays on the plurality of samples to generate the plurality of mass spectrometry datasets.

Embodiment 67. The computer-implemented method of Embodiment 66, wherein the plurality of assays comprises selectively enriching a plurality of chemicals in the plurality of samples.

Embodiment 68. The computer-implemented method of Embodiment 67, wherein the selectively enriching comprises contacting the plurality of samples with a surface.

Embodiment 69. The computer-implemented method of Embodiment 68, wherein the surface comprises a particle surface of a particle.

Embodiment 70. The computer-implemented method of Embodiment 69, wherein the particle comprises a paramagnetic core.

Embodiment 71. The computer-implemented method of any one of Embodiments 67-70, wherein the selectively enriching comprises contacting the plurality of samples with a plurality of surfaces comprising distinct surface chemistries.

Embodiment 72. The computer-implemented method of any one of Embodiments 67-71, wherein the contacting adsorbs the plurality of chemicals on the surface.

Embodiment 73. The computer-implemented method of Embodiment 72, wherein the plurality of chemicals comprises a dynamic range of at least about 1, 2, 3, 4, 5, 6, 7, 8, 9, 10, 11, 12, 13, 14, 15, 16, 17, 18, or 19.

Embodiment 74. The computer-implemented method of Embodiment 72 or 73, wherein the plurality of chemicals comprises a dynamic range of at most about 1, 2, 3, 4, 5, 6, 7, 8, 9, 10, 11, 12, 13, 14, 15, 16, 17, 18, or 19.

Embodiment 75. The computer-implemented method of any one of Embodiments 72-74, wherein the plurality of chemicals, when adsorbed, comprises a dynamic range that is decreased by at least about 1, 2, 3, 4, 5, 6, 7, 8, 9, 10, 11, 12, 13, 14, or 15 magnitudes.

Embodiment 76. The computer-implemented method of any one of Embodiments 72-75, wherein the selectively enriching comprises releasing the plurality of chemicals from the surface.

Embodiment 77. The computer-implemented method of any one of Embodiments 67-71, wherein the plurality of assays comprises performing mass spectrometry on the plurality of samples.

Embodiment 78. The computer-implemented method of any one of Embodiments 1-77, wherein the computing node is a local computing node.

Embodiment 79. The computer-implemented method of Embodiment 78, wherein the plurality of computing nodes comprises at least 2, 5, 10, 100, 1000, 10000, or 100000 computing nodes.

Embodiment 80. The computer-implemented method of Embodiment 78 or 79, wherein the plurality of computing nodes comprises at most 10, 100, 1000, 10000, 100000, or 1000000 computing nodes.

Embodiment 81. The computer-implemented method of any one of Embodiments 1-80, wherein the computing node is a cloud-computing node.

Embodiment 82. The computer-implemented method of any one of Embodiments 1-81, wherein the plurality of computing nodes is a plurality of cloud-computing nodes.

Embodiment 83. The computer-implemented method of any one of Embodiments 1-82, wherein the memory is a cache memory.

Embodiment 84. The computer-implemented method of any one of claims 1-83, wherein the cached dataset is an unserialized cached dataset.

Embodiment 85. The computer-implemented method of Embodiment 84, wherein the unserialized cached dataset is serialized to generate a serialized cached dataset.

Embodiment 86. The computer-implemented method of Embodiment 85, wherein the serialized cached dataset is subdivided to generate a subdivided cached dataset.

Embodiment 87. The computer-implemented method of Embodiment 86, wherein the copy of the cached dataset is a copy of at least a portion of the subdivided cached dataset.

Embodiment 88. The computer-implemented method of Embodiment 87, wherein the transmitting comprises assembling a copy of at least a portion of the serialized cached dataset from the copy of the at least the portion of the subdivided cached dataset.

Embodiment 89. The computer-implemented method of any one of Embodiments 1-88, wherein the cached dataset comprises a pair of mass spectrometry datasets in the plurality of mass spectrometry datasets.

Embodiment 90. The computer-implemented method of Embodiment 89, wherein the transmitting comprises transmitting, to each computing node in the plurality of nodes, a plurality of cached datasets each comprising a unique pair of mass spectrometry datasets in the plurality of mass spectrometry datasets.

Embodiment 91. The computer-implemented method of any one of Embodiments 1-90, wherein the copy of the cached dataset is shared by the plurality of computing nodes.

Embodiment 92. The computer-implemented method of any one of Embodiments 1-91, wherein the plurality of mass spectrometry datasets comprises a plurality of formats.

Embodiment 93. The computer-implemented method of Embodiment 92, further comprising, before (b), generating a harmonized plurality of mass spectrometry datasets comprising a harmonized format based on the plurality of mass spectrometry datasets.

Embodiment 94. The computer-implemented method of Embodiment 93, wherein the loading comprises loading the harmonized plurality of mass spectrometry datasets to generate the cached dataset.

Embodiment 95. The computer-implemented method of Embodiment 94, further comprising, before (b), subdividing each harmonized mass spectrometry datasets in the plurality of mass spectrometry datasets to generate a plurality of mass spectrometry scans.

Embodiment 96. The computer-implemented method of Embodiment 95, wherein the loading comprises loading the plurality of mass spectrometry scans to generate the cached dataset.

Embodiment 97. The computer-implemented method of any one of Embodiments 93-96, wherein the harmonized format comprises a compressed format.

Embodiment 98. The computer-implemented method of any one of Embodiments 93-97, wherein the harmonized format comprises a hierarchical format.

Embodiment 99. The computer-implemented method of any one of Embodiments 93-98, wherein the harmonized format comprises (i) the plurality of mass spectrometry datasets in an indexed series and (ii) indices of the indexed series.

Embodiment 100. The computer-implemented method of any one of Embodiments 95-99, wherein a mass spectrometry dataset in the plurality of mass spectrometry datasets comprises a different number of mass spectrometry scans compared to another mass spectrometry dataset in the plurality of mass spectrometry datasets.

Embodiment 101. The computer-implemented method of any one of Embodiments 93-100, wherein harmonized format is capable of being read in arbitrary slides in the indexed series.

Embodiment 102. The computer-implemented method of any one of Embodiments 93-101, wherein the harmonized format is capable of inserting new datasets and/or being modifyied between arbitrary indices in the indexed series.

Embodiment 103. A computer program product comprising a computer-readable medium having computer-executable code encoded therein, the computer-executable code adapted to be executed to implement any one of the computer-implemented methods of Embodiments 1-102.

Embodiment 104. A non-transitory computer-readable storage media encoded with a computer program including instructions executable by one or more processors to implement any one of the computer-implemented methods of Embodiments 1-102.

Embodiment 105. A computer-implemented system comprising: a digital processing device comprising: at least one processor, an operating system configured to perform executable instructions, a memory, and a computer program including instructions executable by the digital processing device to perform any one of the computer-implemented methods of Embodiments 1-102.

Embodiment 106. A computer-implemented method for performing a plurality of polyamino acid searches based on a plurality of mass spectra and a plurality of user specifications, comprising: (a) displaying a graphical user interface (GUI) to one or more users, wherein the GUI comprises (i) a first menu comprising a plurality of mass spectrum acquisition modes and (ii) a second menu comprising a plurality of mass spectrum search modes; (b) receiving the plurality of user specifications from the one or more users via the GUI, wherein each user specification in the plurality of user specifications comprises (i) a mass spectrum acquisition mode in the plurality of mass spectrum acquisition modes from the first menu and (ii) a mass spectrum search mode in the plurality of mass spectrum search modes from the second menu; (c) receiving the plurality of mass spectra from the one or more users, wherein the plurality of mass spectra comprises a plurality of formats; (d) generating a harmonized plurality of mass spectra based on the plurality of mass spectra and the plurality of formats, wherein the harmonized plurality of mass spectra comprises a harmonized format; and (e) performing the plurality of polyamino acid searches for each mass spectrum in the harmonized plurality of mass spectra based on the plurality of user specifications to generate a plurality of polyamino acid identifications.

Embodiment 107. The computer-implemented method of Embodiment 106, wherein the plurality of mass spectrum acquisition modes comprises data independent acquisition (DIA) and data dependent acquisition (DDA).

Embodiment 108. The computer-implemented method of Embodiment 106 or 107, wherein the plurality of mass spectrum search modes comprises a plurality of DIA search modes.

Embodiment 109. The computer-implemented method of any one of Embodiments 106-108, wherein the plurality of mass spectrum search modes comprises a plurality of DDA search modes.

Embodiment 110. The computer-implemented method of any one of Embodiments 106-109, further comprising performing protein grouping based on the plurality of polyamino acid identifications to generate a plurality of protein groups.

Embodiment 111. The computer-implemented method of Embodiment 110, further comprising displaying a plurality of performance metrics for the plurality of polyamino acid searches, wherein the plurality of performance metrics comprises: (i) a plurality of peptide counts for each mass spectrum in the plurality of mass spectra and (ii) a plurality of protein group counts each mass spectrum in the plurality of mass spectra.

Embodiment 112. The computer-implemented method of Embodiment 111, wherein the plurality of performance metrics comprises a miscleavage rate for each mass spectrum in the plurality of mass spectra.

Embodiment 113. The computer-implemented method of any one of Embodiments 106-112, wherein the performing comprises: subdividing each mass spectrum in the plurality of mass spectra to generate a plurality of mass spectrometry scans; distributing the plurality of mass spectrometry scans onto a plurality of computing nodes; and performing the plurality of polyamino acid searches, using the plurality of computing nodes, to generate the plurality of polyamino acid identifications.

Embodiment 114. The computer-implemented method of Embodiment 113, wherein each mass spectrometry scan in the plurality of mass spectrometry scans comprises a plurality of intensities for a plurality of retention times.

Embodiment 115. The computer-implemented method of Embodiment 113, wherein a first mass spectrometry scan in the plurality of mass spectrometry scans comprises a different mass-to-charge ratio compared to a second mass spectrometry scan in the plurality of mass spectrometry scans.

Embodiment 116. The computer-implemented method of Embodiment 113, further comprising performing mass spectrometry on a plurality of biological samples to generate the plurality of mass spectra.

Embodiment 117. The computer-implemented method of Embodiment 113, wherein the generating further comprises transmitting a first polyamino acid identification of the plurality of polyamino acid identifications from a first computing node in the plurality of computing nodes to a second computing node in the plurality of computing nodes to identify a second polyamino acid identification of the plurality of polyamino acid identifications in the second computing node, wherein the first polyamino acid identification and the second polyamino acid identification are the same.

Embodiment 118. The computer-implemented method of Embodiment 113, wherein the generating further comprises transmitting a probability value associated with a protein group assignment for a polyamino acid identification in the plurality of polyamino acid identifications from a first computing node in the plurality of computing nodes to a second computing node in the plurality of computing nodes.

Embodiment 119. The computer-implemented method of any one of Embodiments 113-118, wherein the plurality of computing nodes is a plurality of cloud-computing nodes.

Embodiment 120. The computer-implemented method of Embodiment 119, wherein the plurality of cloud-computing nodes forms one or more computing clusters.

Embodiment 121. The computer-implemented method of Embodiment 119 or 120, wherein the plurality of cloud-computing nodes forms one or more virtual computing nodes.

Embodiment 122. A computer-implemented method for performing a plurality of polyamino acid searches based on a plurality of mass spectra and a plurality of user specifications, comprising: receiving the plurality of user specifications from the one or more users via a GUI; receiving the plurality of mass spectra from the one or more users, wherein the plurality of mass spectra comprises a plurality of formats; generating a harmonized plurality of mass spectra based on the plurality of mass spectra and the plurality of formats, wherein the harmonized plurality of mass spectra comprises a harmonized format; and performing the plurality of polyamino acid searches for each mass spectrum in the harmonized plurality of mass spectra based on the plurality of user specifications to generate a plurality of polyamino acid identifications.

Embodiment 123. A computer program product comprising a computer-readable medium having computer-executable code encoded therein, the computer-executable code adapted to be executed to implement any one of the computer-implemented methods of Embodiments 106-122.

Embodiment 124. A non-transitory computer-readable storage media encoded with a computer program including instructions executable by one or more processors to implement any one of the computer-implemented methods of Embodiments 106-122.

Embodiment 125. A computer-implemented system comprising: a digital processing device comprising: at least one processor, an operating system configured to perform executable instructions, a memory, and a computer program including instructions executable by the digital processing device to perform any one of the computer-implemented methods of Embodiments 106-122.

Embodiment 126. A computer-implemented system for storing mass spectrometry datasets on a cloud platform, comprising: at least one digital processing device comprising: at least one processor, an operating system configured to perform executable instructions, a memory, and a computer program including instructions that, upon execution by the at least one processor, cause the at least one processor to perform at least: generating an event signal when a mass spectrometry dataset is received by the computer-implemented system, wherein the mass spectrometry dataset comprises at least one of a plurality of formats; triggering an event signal, wherein the event signal instantiates a serverless cloud computing instance; performing a data processing routine using the serverless cloud computing instance, wherein the data processing routine comprises: generating a harmonized mass spectrometry dataset comprising a harmonized data format based on the mass spectrometry dataset; and storing the harmonized mass spectrometry dataset on a storage system.

Embodiment 127. The computer-implemented system of Embodiment 126, wherein the storage system comprises an object-based storage system, a distributed storage system, or an object-based distributed storage system.

Embodiment 128. The computer-implemented system of Embodiment 126 or 127, wherein the harmonized mass spectrometry dataset comprises a columnar format.

Embodiment 129. The computer-implemented system of any one of Embodiments 126-128, wherein instructions further comprise performing the data processing routine using a server cloud computing instance when the serverless cloud computing instance cannot be instantiated.

Embodiment 130. The computer-implemented system of any one of Embodiments 126-129, wherein the data processing routine further comprises (i) performing a plurality of polyamino acid searches based on the harmonized mass spectrometry dataset and a data acquisition mode of the mass spectrometry dataset to generate a plurality of polyamino acid identifications, and (ii) storing the plurality of polyamino acid identifications on the object-based storage system.

Embodiment 131. The computer-implemented system of any one of Embodiments 126-130, wherein the mass spectrometry dataset comprises at least one of a plurality of acquisition modes.

Embodiment 132. The computer-implemented system of any one of Embodiments 126-131, wherein the plurality of acquisition modes comprises data independent acquisition (DIA) and data dependent acquisition (DDA).

Embodiment 133. The computer-implemented system of any one of Embodiments 130-132, wherein the plurality of polyamino acid searches use a plurality of search modes.

Embodiment 134. The computer-implemented system of Embodiment 133, wherein the plurality of search modes comprises a plurality of DIA search modes.

Embodiment 135. The computer-implemented system of Embodiment 133 or 134, wherein the plurality of search modes comprises a plurality of DDA search modes.

Embodiment 136. The computer-implemented system of any one of Embodiments 126-135, wherein the data processing routine further comprises performing protein grouping based on the plurality of polyamino acid identifications to generate a plurality of protein groups.

Embodiment 137. The computer-implemented system of Embodiment 136, wherein the performing the protein grouping comprises: (i) subdividing the harmonized mass spectrometry dataset to generate a plurality of mass spectrometry scans; (ii) distributing the plurality of mass spectrometry scans onto a plurality of computing nodes; and (iii) performing the plurality of polyamino acid searches, using the plurality of computing nodes, to generate the plurality of protein groups.

Embodiment 138. The computer-implemented system of any one of Embodiments 126-137, wherein each mass spectrometry scan in the plurality of mass spectrometry scans comprises a plurality of intensities for a plurality of retention times.

Embodiment 139. A computer-implemented method for storing mass spectrometry datasets on a cloud platform, comprising: (a) receiving a mass spectrometry dataset, wherein the mass spectrometry dataset comprises at least one of a plurality of formats; (b) generating an event signal based on the mass spectrometry dataset; (c) instantiating a serverless cloud computing instance based on the event signal; (d) performing a data processing routine using the serverless cloud computing instance, wherein the data processing routine comprises: (i) generating a harmonized mass spectrometry dataset comprising a harmonized data format based on the mass spectrometry dataset; and (ii) storing the harmonized mass spectrometry dataset on an object-based storage system.

Embodiment 140. The computer-implemented method of Embodiment 139, wherein the harmonized mass spectrometry dataset comprises a columnar format.

Embodiment 141. The computer-implemented method of Embodiment 139 or 140, further comprising performing the data processing routine using a server cloud computing instance when the serverless cloud computing instance cannot be instantiated.

Embodiment 142. The computer-implemented method of any one of Embodiments 139-141, wherein the data processing routine further comprises (i) performing a plurality of polyamino acid searches based on the harmonized mass spectrometry dataset and a data acquisition mode of the mass spectrometry dataset to generate a plurality of polyamino acid identifications, and (ii) storing the plurality of polyamino acid identifications on the object-based storage system.

Embodiment 141. The computer-implemented method of any one of Embodiments 139-140, wherein the mass spectrometry dataset comprises at least one of a plurality of acquisition modes.

Embodiment 142. The computer-implemented method of Embodiment 141, wherein the plurality of acquisition modes comprises data independent acquisition (DIA) and data dependent acquisition (DDA).

Embodiment 143. The computer-implemented method of any one of Embodiments 139-142, wherein the plurality of polyamino acid searches use a plurality of search modes.

Embodiment 144. The computer-implemented method of Embodiment 143, wherein the plurality of search modes comprises a plurality of DIA search modes.

Embodiment 145. The computer-implemented method of Embodiment 143 or 144, wherein the plurality of search modes comprises a plurality of DDA search modes.

Embodiment 146. The computer-implemented method of any one of Embodiments 139-145, wherein the data processing routine further comprises performing protein grouping based on the plurality of polyamino acid identifications to generate a plurality of protein groups.

Embodiment 147. The computer-implemented method of Embodiment 146, wherein the performing the protein grouping comprises: (i) subdividing the harmonized mass spectrometry dataset to generate a plurality of mass spectrometry scans; (ii) distributing the plurality of mass spectrometry scans onto a plurality of computing nodes; and (iii) performing the plurality of polyamino acid searches, using the plurality of computing nodes, to generate the plurality of protein groups.

Embodiment 148. The computer-implemented method of any one of Embodiments 139-147, wherein each mass spectrometry scan in the plurality of mass spectrometry scans comprises a plurality of intensities for a plurality of retention times.

Embodiment 149. The computer-implemented method of any one of Embodiments 139-148, further comprising (a) receiving a second mass spectrometry dataset; (b) generating a second event signal based on the mass spectrometry dataset; (c) instantiating a second serverless cloud computing instance based on the event signal; (d) performing a second data processing routine based on the second mass spectrometry dataset using the second serverless cloud computing instance, wherein the data processing routine and the second data processing routine are performed in parallel.

Embodiment 150. A computer-implemented method for processing a mass spectrometry (MS) dataset to store a trace in a distributed storage system: (a) extracting a plurality of signals from the MS dataset, wherein each signal in the plurality of signals comprises a mass-to-charge ratio (m/z), a retention time, and an intensity, wherein the plurality of signals is extracted when the m/z of a signal in the MS dataset is within a predetermined range from a reference m/z of a reference feature in the MS dataset; and (b) storing the trace comprising the plurality of signals in association with an identifier for the reference feature in the distributed storage system.

Embodiment 151. The computer-implemented method of Embodiment 150, wherein the reference feature is annotated with a polyamino acid.

Embodiment 152. The computer-implemented method of Embodiment 150 or 151, wherein the MS dataset comprises a columnar format.

Embodiment 153. The computer-implemented method of any one of Embodiments 150-152, further comprising loading the MS dataset to a plurality of cache memories of a distributed computing system to generate a cached dataset.

Embodiment 154. The computer-implemented method of Embodiment 153, further comprising storing the cached dataset in the distributed storage system.

Embodiment 155. The computer-implemented method of Embodiment 153 or 154, wherein the cached dataset is stored in a columnar format.

Embodiment 156. The computer-implemented method of any one of Embodiments 153-155, wherein the cached dataset is stored in a binary format.

Embodiment 157. The computer-implemented method of any one of Embodiments 154-156, further comprising loading the cached dataset from the distributed storage system.

Embodiment 158. The computer-implemented method of any one of Embodiments 150-157, wherein the distributed storage system comprises an object-based storage system.

Embodiment 159. The computer-implemented method of any one of Embodiments 150-158, further comprising loading the trace into a plurality of cache memories of a distributed computing system.

Embodiment 160. The computer-implemented method of any one of Embodiments 150-159, further comprising displaying the trace on a graphical user interface.

Embodiment 161. The computer-implemented method of any one of Embodiments 150-160, further comprising, before (a), identifying the reference feature in the MS dataset.

Embodiment 162. The computer-implemented method of any one of Embodiments 150-161, further comprising, before (a), identifying a plurality of reference features in the MS dataset.

Embodiment 163. The computer-implemented method of any one of Embodiments 150-162, further comprising extracting a second plurality of signals from the MS dataset based on a second reference feature in the MS dataset.

Embodiment 164. The computer-implemented method of Embodiment 163, wherein the extracting the plurality of signals and the second plurality of signals is performed in parallel.

Embodiment 165. The computer-implemented method of Embodiment 163 or 164, further comprising storing a second trace comprising the second plurality of signals in association with a second identifier for the second reference feature in the distributed storage system.

Embodiment 166. The computer-implemented method of any one of Embodiments 163-166, wherein the storing the plurality of signals and the second plurality of signals is performed in parallel.

Embodiment 167. A computer program product comprising a computer-readable medium having computer-executable code encoded therein, the computer-executable code adapted to be executed to implement any one of the computer-implemented methods of Embodiments 139-166.

Embodiment 168. A non-transitory computer-readable storage media encoded with a computer program including instructions executable by one or more processors to implement any one of the computer-implemented methods of Embodiments 139-166.

Embodiment 169. A computer-implemented system comprising: a digital processing device comprising: at least one processor, an operating system configured to perform executable instructions, a memory, and a computer program including instructions executable by the digital processing device to perform any one of the computer-implemented methods of Embodiments 139-166.

Embodiment 170. A method for identifying protein groups, comprising: (a) obtaining a plurality of independently measured mass spectrometry data; (b) subdividing each mass spectrometry data in the plurality of independently measured mass spectrometry data to provide a set of elements; (c) distributing the set of elements onto a plurality of nodes; and (d) generating, using the plurality of nodes, identifications of one or more biomolecules based at least in part on the set of elements.

Embodiment 171. The method of Embodiment 170, wherein the plurality of independently measured mass spectrometry data comprises mass spectrometry data obtained by performing mass spectrometry on a plurality of biological samples.

Embodiment 172. The method of Embodiment 170, wherein the plurality of nodes comprises a distributed computing system.

Embodiment 173. The method of Embodiment 172, wherein the set of elements comprise a set of mass spectrometry scans.

Embodiment 174. The method of Embodiment 173, wherein a first node in the plurality of nodes is configured to transfer one or more annotations in a first mass spectrometry scan to a second node in the plurality of nodes.

Embodiment 175. The method of Embodiment 174, wherein the identifications comprise one or more peptide spectral matches.

Embodiment 176. The method of Embodiment 172, wherein the set of elements comprise a set of peptide identifications.

Embodiment 177. The method of Embodiment 176, wherein a first node in the plurality of nodes is configured to transfer one or more probability values associated with a protein group assignment for one or more peptide identifications in the set of peptide identifications to a second node in the plurality of nodes.

Embodiment 178. The method of Embodiment 177, wherein the identifications comprise one or more protein group identifications.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1-33. (canceled)

34. A computer-implemented method for normalizing and processing mass spectrometry datasets, comprising:
(a) obtaining a plurality of mass spectrometry datasets obtained from a plurality of samples;
(b) loading the plurality of mass spectrometry datasets into a memory of a computing node to generate a cached dataset;
(c) transmitting a copy of the cached dataset to a plurality of cache memories of a plurality of computing nodes;
(d) determining, using the plurality of computing nodes, a plurality of feature values for the plurality of mass spectrometry datasets;
(e) normalizing, using the plurality of computing nodes, across the plurality of mass spectrometry datasets using the plurality of feature values to generate a plurality of normalized mass spectrometry datasets; and
(f) processing the plurality of normalized mass spectrometry datasets to compare the plurality of samples.

35. The computer-implemented method of claim 34, wherein the plurality of mass spectrometry datasets comprises a set of polyamino acid identifications and a set of polyamino acid intensities for each sample in the plurality of samples.

36. The computer-implemented method of claim 35, wherein the normalizing generates a set of aligned precursors for each mass spectrometry dataset in the plurality of mass spectrometry datasets.

37. The computer-implemented method of claim 35, wherein the normalizing generates a set of relative abundances for each mass spectrometry dataset in the plurality of mass spectrometry datasets.

38. The computer-implemented method of claim 35, wherein the normalizing comprises adjusting the set of polyamino acid intensities for each mass spectrometry dataset in the plurality of mass spectrometry datasets based on the plurality of feature values.

39. The computer-implemented method of claim 34, wherein the normalizing comprises minimizing an objective function for a unique pair of mass spectrometry datasets in the plurality of mass spectrometry datasets for each computing node in the plurality of computing nodes.

40. The computer-implemented method of claim 34, wherein the processing further comprises determining a biomarker based on the plurality of normalized mass spectrometry datasets.

41. The computer-implemented method of claim 34, wherein the processing further comprises training a machine learning model based on the plurality of normalized mass spectrometry datasets.

42. The computer-implemented method of claim 34, wherein the cached dataset is an unserialized cached dataset.

43. The computer-implemented method of claim 42, wherein the unserialized cached dataset is serialized to generate a serialized cached dataset.

44. The computer-implemented method of claim 43, wherein the serialized cached dataset is subdivided to generate a subdivided cached dataset.

45. The computer-implemented method of claim 44, wherein the copy of the cached dataset is a copy of at least a portion of the subdivided cached dataset.

46. The computer-implemented method of claim 45, wherein the transmitting comprises assembling a copy of at least a portion of the serialized cached dataset from the copy of the at least the portion of the subdivided cached dataset.

47. The computer-implemented method of claim 34, wherein the transmitting comprises transmitting, to each computing node in the plurality of nodes, a plurality of cached datasets each comprising a unique pair of mass spectrometry datasets in the plurality of mass spectrometry datasets.

48. The computer-implemented method of claim 34, wherein the copy of the cached dataset is shared by the plurality of computing nodes.

49. The computer-implemented method of claim 34, wherein the plurality of mass spectrometry datasets comprises a plurality of formats.

50. The computer-implemented method of claim 49, further comprising, before (b), generating a harmonized plurality of mass spectrometry datasets comprising a harmonized format based on the plurality of mass spectrometry datasets.

51. The computer-implemented method of claim 50, further comprising, before (b), subdividing each harmonized mass spectrometry datasets in the plurality of mass spectrometry datasets to generate a plurality of mass spectrometry scans.

52. The computer-implemented method of claim 50, wherein the harmonized format comprises (i) the plurality of mass spectrometry datasets in an indexed series and (ii) indices of the indexed series, and wherein the harmonized format is capable of being read in arbitrary slices in the indexed series and is capable of inserting new datasets and/or being modified between arbitrary indices in the indexed series.

53. A computer-implemented method for normalizing and processing mass spectrometry datasets, comprising:
(a) obtaining a plurality of mass spectrometry datasets obtained from a plurality of samples;
(b) generating a harmonized plurality of mass spectrometry datasets comprising a harmonized format based on the plurality of mass spectrometry datasets, wherein the harmonized format comprises (i) the plurality of mass spectrometry datasets in an indexed series and (ii) indices of the indexed series, such that the harmonized format is capable of being read in arbitrary slices in the indexed series and of inserting new datasets and/or being modified between arbitrary indices in the indexed series;
(c) loading the harmonized plurality of mass spectrometry datasets into a memory of a computing node to generate a cached dataset;
(d) transmitting a copy of the cached dataset to a plurality of cache memories of a plurality of computing nodes;
(e) determining, using the plurality of computing nodes, a plurality of feature values for the harmonized plurality of mass spectrometry datasets;
(f) normalizing, using the plurality of computing nodes, across the harmonized plurality of mass spectrometry datasets using the plurality of feature values to generate a harmonized plurality of normalized mass spectrometry datasets; and
(g) processing the harmonized plurality of normalized mass spectrometry datasets to compare the plurality of samples.
